(19) United States
(12) Patent Application Publication
(10) Pub. No.: US 2014/0011702 A1
(43) Pub. Date: Jan. 9, 2014

(54) BIOMARKERS AND METHODS FOR THE PROGNOSIS OF GLIOBLASTOMA

(71) Applicants: Jean Mosser, Rennes (FR); Amandine Etcheverry, Rennes (FR); Marc Aubry, Rennes (FR); Jean-Yves Delattre, Charenton (FR)

(72) Inventors: Jean Mosser, Rennes (FR); Amandine Etcheverry, Rennes (FR); Marc Aubry, Rennes (FR); Jean-Yves Delattre, Charenton (FR)

(73) Assignee: CENTRE HOSPITALIER UNIVERSITAIRE PONTCHAILLOU, Rennes (FR)

(21) Appl. No.: 14/006,023

(22) PCT Filed: Dec. 13, 2011
(86) PCT No.: PCT/EP2011/072641

§ 371 Date: Sep. 18, 2013

Related U.S. Application Data
(60) Provisional application No. 61/466,672, filed on Mar. 23, 2011.
(60) Provisional application No. 61/515,025, filed on Aug. 4, 2011.

(51) Int. Cl. C12Q 1/68

(52) U.S. Cl. CPC C12Q 1/6886
USPC 506/9; 435/6.11

(57) ABSTRACT

The present invention relates to gene promoters whose methylation status correlates with the clinical survival outcome of glioblastoma patients treated according to the Stupp protocol. More specifically, the invention provides methods and kits for the prognosis of survival outcome and/or treatment response in glioblastoma patients.

### RELATED APPLICATION

The present patent application claims priority to U.S. Provisional Patent Application No. 61/466,672 filed on Mar. 23, 2011 and U.S. Provisional Patent Application No. 61/515,025 filed on Aug. 4, 2011. The entire content of each of the Provisional patent applications is incorporated herein by reference in its entirety.

### BACKGROUND OF THE INVENTION

Glioblastoma is the most common and aggressive primary brain tumor in adults. Its prognosis remains extremely poor, despite multimodal treatment by surgery, radiotherapy and chemotherapy (Wen et al., N Engl J Med 2008, 359: 492-507). These tumors are now well characterized at the transcriptome and genome levels. Several studies have demonstrated that a combination of these two molecular levels may be advantageous for determining robust signatures and clinically relevant molecular classifiers of glioblastoma (de Tayrac et al., Genes Chromosomes Cancer 2009, 48: 55-68; Nigro et al., Cancer Res, 2005, 65: 1678-1686).

The role of general epigenetic mechanisms in carcinogenesis and tumor aggressiveness is well documented: CpG island hypermethylation silences tumor suppressor genes, whereas hypomethylation promotes the transcriptional activation of oncogenes and induces chromosomal instability (Herman et al., N Engl J Med, 2003, 349: 2042-2054; Karpf et al., Cancer Res, 2005, 65: 8635-8639). Such epigenic changes are potentially reversible and may therefore be considered promising targets for epigenetic anticancer treatments. Indeed, the use of DNA-demethylating drugs (5-azacytidine and 5-aza-2′-deoxicytidine) has been approved by the Food and Drug Administration (FDA) for the treatment of myelodysplastic syndromes and myelogenous leukemia (Garcia-Manero, Curr Opin Oncol, 2008, 20: 705-710; Mack, J Natl Cancer Inst, 2006, 98: 1443-1444).

A few clinically-relevant biomarkers have been identified so far in glioblastoma. The somatic mutation affecting amino acid 132 of the isocitrate dehydrogenase 1 (IDH1) gene is an independent prognostic biomarker associated with better clinical outcome in gliomas, including glioblastoma (Ichimura et al., Neuro Oncol, 2009, 11: 341-347; Yan et al., N Engl J Med, 2009, 360: 765-773), but this mutation is rare in glioblastoma (around 6%) and concerns almost exclusively secondary glioblastomas (Sanson et al., J Clin Oncol, 2009, 274150-4154). Changes in promoter DNA methylation pattern of genes involved in key biological pathways have been reported in glioblastoma. For instance, the retinoblastoma (RB), the PI3K and p53 pathways are affected by CpG island promoter hypermethylation (RB, CDKN2A, PTEN, TP53) (Watanabe et al., J Neuropathol Exp Neurol, 2001, 60: 1181-1189; Nakamura et al., Lab Invest, 2001, 81: 77-82; Costello et al., Cancer Res, 1996, 56: 2405-2410; Bello et al., Cancer Genet Cytogenet, 2006, 164: 172-173; Amatya et al., Acta Neuropathol, 2005, 110: 178-184). Epigenic silencing of the 06-methylguanine DNA methyltransferase (MGMT) gene, which encodes a DNA repair enzyme, sensitizes cancer cells to alkylating agents, and is associated with significantly longer survival in glioblastoma patients treated with the standard treatment known as “the Stupp Protocol”, which includes surgery, radiotherapy and concomitant and adjuvant temozolomide (Stupp et al., Lancet Oncol, 2009, 10: 459-466). The methylation status of the MGMT promoter is believed to be the strongest predictor of outcome and benefit from the temozolomide treatment (Gorlia et al., Lancet Oncol, 2008, 9: 29-38).

Genome-wide assessments of DNA methylation are now necessary to decipher the epigenetic events involved in the aggressive phenotype of glioblastoma and to guide new treatment strategies. Several microarray-based glioblastoma studies have identified gene promoters that are frequently hyper- or hypomethylated. These gene promoters were initially identified indirectly, via pharmacologic or RNAi-induced inhibition of DNA methyltransferase in glioblastoma cell lines (Kim et al., Cancer Res, 2006, 66: 7490-7501; Foltz et al., Oncogene, 2009, 28: 2667-2677), or by using methyl-CpG-binding proteins (Wu et al., Cancer Res, 2010, 70: 2718-2727). More recently, direct hybridization of bisulfite-modified DNA on beadchips has made it possible to reliably quantify promoter methylation (Martinez et al., Epigenetics, 2009, 4: 255-264; Noushmehr et al., Cancer Cell, 2010, 17: 510-522) in cohorts of patients. Noushmehr et al. used this technique to profile DNA methylation alterations in 272 glioblastomas in the context of The Cancer Genome Altas (TCGA). They reported a rare subgroup of glioblastomas displaying a concerted multilocus hypermethylation pattern and suggested the existence of a Glioma CpG Island Methylator Phenotype (G-CIMP). G-CIMP tumors tended to be secondary and recurrent glioblastomas, and were tightly associated with IDH1 somatic mutation.

Therefore, there still exists a great need in the art for new biological markers of glioblastoma, in particular biomarkers with improved predictive performance over the standard MGMT promoter methylation status method.

### SUMMARY OF THE INVENTION

The present invention generally relates to improved systems and strategies for the prognosis of survival outcome of glioblastoma patients. In particular, the invention provides biomarkers and methods that improve the conventional MGMT stratification of glioblastoma patients. Indeed, the present Applicants have performed a methylome-based survival analysis of one of the largest uniformly treated (radiotherapy and chemotherapy with concomitant and adjuvant temozolomide) glioblastoma cohort ever studied, for more than 27,000 CpG sites. In this cohort, they identified 60 di-nucleotide CpG sites (in addition to the MGMT promoter methylation status) that were significantly associated with clinical overall survival (see Example 1). Such promoter CpG sites can constitute new epigenetic prognostic markers in glioblastoma. In addition, to test whether these epigenetic markers may improve the molecular prognostic stratification of glioblastoma, the Applicants have investigated a series of 233 glioblastoma patients treated with the standard Stupp regimen. They showed that the methylation status of the DGKI promoter and SDPR promoter modulates the prognostic value of the MGMT promoter methylation status in glioblastoma patients.

Accordingly, in one aspect, the present invention provides an in vitro method for providing a prognosis for a patient diagnosed with glioblastoma, the method comprising steps of: determining, in a biological sample obtained from the patient, the methylation status of the DGKI promoter and the MGMT promoter, or the DGKI promoter, the SDPR promoter and the MGMT promoter, to obtain a gene promoter methylation pattern for the sample, and based on the gene promoter methylation pattern obtained, providing a prognosis for the patient, wherein the prognosis comprises the survival outcome of the patient, if the patient were to be treated according to the Stupp protocol.

In such a method, the simultaneous hypermethylation of the MGMT promoter and hypermethylation of the DGKI promoter, or the simultaneous hypomethylation of the MGMT promoter and hypomethylation of the SDPR promoter, is indicative of a short-term survival outcome for the patient, if the patient were to be treated according to the Stupp protocol.

In such as method, the simultaneous hypermethylation of the MGMT promoter, hypomethylation of the DGKI promoter and hypomethylation of the SDPR promoter, or the simultaneous hypomethylation of the MGMT promoter and hypermethylation of the SDPR promoter, is indicative of a medium-term survival outcome for the patient, if the patient were to be treated according to the Stupp protocol.

In such a method, the simultaneous hypermethylation of the MGMT promoter, hypomethylation of the DGKI promoter and hypermethylation of the SDPR promoter is indicative of a long-term survival outcome for the patient, if the patient were to be treated according to the Stupp protocol.

In certain embodiments, the method further comprises a step of prescribing a treatment to the glioblastoma patient based on the prognosis provided. Prescribing a treatment may be prescribing a treatment according to the Stupp protocol, prescribing an alternative treatment to the Stupp protocol, or including the patient in a glioblastoma clinical trial.

The present invention also provides an in vitro method for providing a prognosis for a patient diagnosed with glioblastoma, the method comprising steps of: determining, in a biological sample obtained from the patient, the methylation status of a promoter of at least one gene selected from the group consisting of TBX3, FSD1, FNDC3B, DGKI, AGT, FLJ25422, SEPP1, SOX10, MAP3K14, ACOT8, KCNMB1, CHI3L2, COG4, FAM49A, GPR85, CCND1, MGC29671, LGALS1, SDPR, GPR128, NET1, SLC26A5, RNASE3, CDKN2B, NUP98, CYP24A1, ACTL6B, KLK10, TRPV4, CX36, TRIM58, GRIP1, PHLDA2, PON1, SLC2A2, TNF, FLJ23657, C1orf176, FLJ32447, HOXA11, LY6K, HMG20B, KHDRBS2, WT1, TFF2, ZNF542, ZSCAN1, ZNF540, HBZ, GPR92, HOXA9, KCNA4, RAC2, CYP1B1, FUT3, GCET2, MEGF10, GRK1, GPX5, and any combination thereof, to obtain a gene promoter methylation pattern for the sample, and based on the gene promoter methylation pattern obtained, providing a prognosis for the patient, wherein the prognosis comprises the expected response of the patient to the treatment, if the patient were to be treated according to the Stupp protocol.

In such a method, hypomethylation of SOX10 promoter is indicative of a patent who would be responsive to the Stupp treatment, if the patient were to be treated according to the Stupp protocol.

In such a method, the simultaneous hypermethylation of the MGMT promoter and one or more of: hypomethylation of the FNDC3B promoter, hypermethylation of the TBX3 promoter, hypermethylation of the DGKI promoter, and hypermethylaltion of the FSD1 promoter, is indicative of a patent who would be non-responsive to the Stupp treatment, if the patient were to be treated according to the Stupp protocol.

In certain embodiments, the method further comprises a step of prescribing a treatment to the glioblastoma patient based on the prognosis provided. Prescribing a treatment may be prescribing a treatment according to the Stupp protocol, prescribing an alternative treatment to the Stupp protocol, or including the patient in a glioblastoma clinical trial.

In the prognostic methods of the invention, the biological sample generally genomic DNA that may be extracted from a fresh tissue sample, a frozen tissue sample or a fixed, paraffin-embedded tissue sample. In certain preferred embodiments, the tissue sample is glioblastoma tissue obtained during surgery.

In the prognostic methods of the invention, determining the methylation status is performed after sodium bisulfite conversion of the extracted genomic DNA and may comprise using direct sequencing, pyrosequencing, Combined Bisulfite Restriction Analysis (COBRA), Methylation-Sensitive Single-Nucleotide Primer Extension (MS-SnuPE), Methylation-Sensitive Melting Curve Analysis or (MS-MSA), Methylation-Sensitive High-Resolution Melting (MS-HRM), MALDI-TOF mass spectrometry, HeavyMethyl, methylation specific PCR (MSP), MethylLight, Melting curve Methylation Specific PCR (McMSP), Sensitive Melting Analysis after Real-Time MSP (SMART-MSP), Methylation-Specific Fluorescent Amplicon Generation (MS-FLAG) or any combination thereof.

In another aspect, the present invention provides a kit for the in vitro prognosis of a glioblastoma patient treated in accordance with the Stupp protocol, the kit comprising methylation-specific PCR (MSP) primers, methylation-independent PCR (MIP) primers or pyrosequencing primers to detect the methylation status of the DGKI promoter, the SDPR promoter and/or the MGMT promoter in genomic DNA after sodium bisulfite conversion.

The present invention also provides a kit for the in vitro prognosis of a glioblastoma patient treated in accordance with the Stupp protocol, the kit comprising methylation-specific PCR (MSP) primers, methylation-independent PCR (MIP) primers or pyrosequencing primers to detect the methylation status of at least one gene promoter in genomic DNA after sodium bisulfite conversion, wherein the gene promoter is TBX3, FSD1, FNDC3B, DGKI, AGT, FLJ25422, SEPP1, SOX10, MAP3K14, ACOT8, KCNMB1, CHI3L2, COG4, FAM49A, GPR85, CCND1, MGC29671, LGALS1, SDPR, GPR128, NET1, SLC26A5, RNASE3, CDKN2B, NUP98, CYP24A1, ACTL6B, KLK10, TRPV4, CX36, TRIM58, GRIP1, PHLDA2, PON1, SLC2A2, TNF, FLJ23657, C1orf176, FLJ32447, HOXA11, LY6K, HMG20B, KHDRBS2, WT1, TFF2, ZNF542, ZSCAN1, ZNF540, HBZ, GPR92, HOXA9, KCNA4, RAC2, CYP1B1, FUT3, GCET2, MEGF10, GRK1, GPX5 or any combination thereof.

In certain embodiments, the at least one gene promoter is the SOX10 promoter.

In other embodiments, the at least one gene promoter is the FNDC3B promoter, and/or the TBX3 promoter, and/or the DGKI promoter, and/or the FSD1 promoter.

In certain embodiment, the kit may further comprise methylation-specific PCR (MSP) primers, methylation-independent PCR (MIP) primers or pyrosequencing primers to detect the methylation status of the MGMT promoter.

These and other objects, advantages and features of the present invention will become apparent to those of ordinary skill in the art having read the following detailed description of the preferred embodiments.

### BRIEF DESCRIPTION OF THE DRAWING

FIG. 1 is a graph showing the maximal gene expression mean values by β-value bins (5% wide), in glioblastomas (n=40). The expression values presented are normalized and log-transformed intensities. Error bars are also shown. The grey rectangles define the β-value ranges for which a change in maximal expression values is observed.

FIG. 2 shows the hypermethylated CpGs located within PRC2-targeted promoters. (A) Heatmap of the hypermethylated CpGs located within PRC2-targeted promoters. Samples are ranked horizontally as a function of their mean β-values. Two clusters representing extreme methylation changes (Δβ) relative to control samples (N) are framed. (B) EZH2 and DNMT3A expression level in control samples, glioblastoma samples, the low- and the high-Δβ clusters. The expression values presented are normalized and log-transformed intensities.

FIG. 3 shows the expression and methylation profiles of the SERPINB1 gene in glioblastoma patients (n=40). The expression values presented are normalized and log-transformed intensities. The methylation values are β-values.

FIG. 4 shows results of a Kaplan-Meier estimation of the overall survival in 50 glioblastomas treated in accordance with the STUPP protocol. Patients were assigned to groups according to the methylation status of (A) MGMT, (B) SOX10 site #2, (C) MGMT and FNDC3B, and (D) MGMT and TBX3. M: methylated; NM: non-methylated. P-values for the difference in OS (log-rank test), size and median survival of each group are also reported. See Table 3 for β-values cut-offs.

FIG. 5 shows a survival associated CpG sites NMF classification. (A) Cophenetic correlation and dispersion coefficients for two to ten class decomposition of the survival associated CpG sites methylation profiles (solid line) and randomized data (dashed line). (B) Reordered consensus matrix computed at k=3 for the survival associated CpG sites showing three stable methylation classes.

FIG. 6 shows a Kaplan-Meier estimation of Overall Survival (OS). (A) All patients, MGMT methylation status. (B) MGMT-methylated patients, DGKI_{—}7 methylation status. (C) MGMT-methylated patients, DGKI_{—}7 and SDPR methylation status. (D) MGMT-unmethylated patients, DGKI_{—}7 and SDPR methylation status. (M: methylated; UM: unmethylated, mo: month). Difference in OS (log-rank test p-value), size and median survival of each group are reported.

FIG. 7 shows the results of a tri-methylation category. (A) Patient tri-methylation category is defined by the combination of its tumor methylation status for MGMT_{—}1, DGKI_{—}7 and SDPR (STS: short-term survival, MTS: mid-term survival, and LTS: long-term survival). Grey boxes: the methylation status of the marker does not contribute to the stratification. (B) Kaplan-Meier estimation of OS and patient tri-methylation category. (C) Kaplan-Meier estimation of PFS and patient tri-methylation category. (M: methylated; UM: unmethylated, mo: month. Difference in survival (log-rank test p-value), size and median survival of each group are reported).

FIG. 8 shows a Kaplan-Meier estimation of survival for MGMT-methylated and IDH1 wild-type patients.

### DEFINITIONS

Throughout the specification, several terms are employed that are defined in the following paragraphs.

The terms “subject” and “individual” are used herein interchangeably. They refer to a human being who may or may not suffer from glioblastoma (GBM). In many embodiments of the present invention, the subject has been diagnosed with GBM. In such embodiments, the subject may also be called “patient”. The terms “subject”, “individual” and “patient” do not denote a particular age.

As used herein, the term “Stupp protocol” refers to the treatment regimen that is the current standard of care for glioblastoma patients, and which consists of surgery to remove the maximal amount of tumor followed by radiotherapy and chemotherapy comprising concomitant and adjuvant temozolomide, as described by Stupp and coworkers (Lancet Oncol, 2009, 10: 459-466).

The term “biomarker” refers to a substance that is a distinctive indicator of a biological process, biological event and/or pathological condition. In the context of the present invention, the methylation status of a biomarker of the invention is, alone or in combination with the methylation status of other biomarkers, a distinctive indicator of the prognosis of glioblastoma patients, if the patients were to be treated according to the Stupp protocol, and in particular a distinctive indicator of the clinical survival outcome of such patients. In the methods of the invention, prognosis of the survival outcome of such patients includes determination of the methylation status of a gene promoter.

As used herein, the term “gene” refers to a polynucleotide that encodes a discrete macromolecular product, be it a RNA or a protein, and may include regulatory sequences preceding (5′ non-coding sequences) and following (3′ non-coding sequences) the coding sequence. As more than one polynucleotide may encode a discrete product, the term also includes alleles and polymorphisms of a gene that encode the same product, or a functionally associated (including gain, loss, or modulation of function) analog thereof.

As used herein, the term “gene promoter” has its art understood meaning, and refers to a region of DNA that facilitates the transcription of a particular gene. More specifically, a promoter is a region of DNA typically extending 150-300 bp upstream from the transcription start site of the gene, and which contains binding sites for RNA polymerase and for a number of proteins that regulate the rate of transcription of the adjacent gene. A method of prognosis according to the present invention generally includes determination of the methylation status of one or more CpG sites of a gene promoter.

As used herein the term “CpG site” refers to a region of DNA where a cytosine nucleotide occurs next to a guanine nucleotide in the linear sequence of bases along its length, the cytosine (C) being separated by only one phosphate (p) from the guanine (G). About 70% of human gene promoters have a high CpG content. Regions of the genome that have a higher concentration of CpG sites are known as “CpG islands”. Cytosines in CpG dinucleotides can be methylated to form 5-methylcytosine. Methylation of (i.e., introduction of a methyl group in) the cytosines of CpG site within the promoters of genes can lead to gene silencing, a feature found in a number of human cancers. In contrast, the hypomethylation of CpG sites has generally been associated with the over-expression of oncogenes within cancer cells.

The term “methylation status”, as used herein to describe the state of methylation of a gene promoter, refers to the presence or absence of 5-methylcytosine at one CpG site within a gene promoter. When none of the DNA of an individual is methylated at one given CpG site, the gene promoter is 0% methylated. When all the DNA of the individual is methylated at that given CpG site, the gene promoter is 100% methylated. When only of portion, e.g., 50%, 75%, or 80%, of the DNA of the individual is methylated at that CpG site, then the gene promoter is said to be 50%, 75%, or 80%, methylated, respectively. The term “methylation status” reflects any relative or absolute amount of methylation of a gene promoter. Methylation of gene promoters can be assessed by any method used in the art. The terms “methylation” and “hypermethylation” are used herein interchangeably. When used in reference to a gene promoter, they refer to the methylation status corresponding to an increased presence of 5-methylcytosine at a CpG site within the gene promoter of a biological sample obtained from a glioblastoma patient, relative to the amount of 5-methylcytosine found at the CpG site within the same gene promoter of a biological sample obtained from a healthy individual. In certain embodiments, a gene promoter is considered to be methylated or hypermethylated if its methylation status is higher than a threshold value, e.g. a threshold value determined by the present Applicants. The terms “unmethylation”, “non-methylation” and “hypomethylation” are used herein interchangeably. When used in reference to a gene promoter, they refer to the methylation status corresponding to a decreased presence of 5-methylcytosine at a CpG site within the gene promoter of a biological sample obtained from a glioblastoma patient, relative to the amount of 5-methylcytosine found at the CpG site within the same gene promoter of a biological sample obtained from a healthy individual. In certain embodiments, a gene promoter is considered to be unmethylated or hypomethylated if its methylation status is lower than a threshold value, e.g. a threshold value determined by the present Applicants.

The term “biological sample” is used herein in its broadest sense. In the practice of the present invention, a biological sample is generally obtained from a subject. A sample may be any biological tissue or fluid with which the methylation status of biomarkers of the present invention may be assayed. Frequently, a sample will be a “clinical sample” (i.e., a sample obtained or derived from a patient to be tested). The sample may also be an archival sample with known diagnosis, treatment, and/or outcome history. Examples of biological samples suitable for use in the practice of the present invention include, but are not limited to, bodily fluids, e.g., blood samples (e.g., blood smears), and cerebrospinal fluid, brain tissue samples or bone marrow tissue samples such as tissue or fine needle biopsy samples. Biological samples may also include sections of tissues such as frozen sections taken for histological purposes. The term “biological sample” also encompasses any material derived by processing a biological sample. Derived materials include, but are not limited to, cells (or their progeny) isolated from the sample, as well as nucleic acid molecules (DNA and/or RNA) extracted from the sample. Processing of a biological sample may involve one or more of: filtration, distillation, extraction, concentration, inactivation of interfering components, addition of reagents, and the like.

The terms “normal” and “healthy” are used herein interchangeably. They refer to a subject who has not been diagnosed with glioblastoma and, more generally, who is known to display a non-neoplastic brain. In certain embodiments, normal subjects may have similar age, body mass index and/or sex as compared with the patient from whom the biological sample to be tested was obtained. The term “normal” is also used herein to qualify a sample obtained from a healthy subject.

In the context of the present invention, the term “control”, when used herein to characterize a subject, refers to a subject who is healthy or to a patient who has been diagnosed with a specific disease (in particular a brain disease or disorder, such as brain tumors) other than glioblastoma, or yet to a patient diagnosed with glioblastoma and treated according to the Stupp protocol and whose clinical survival outcome is known. The term “control sample” refers to one, or more than one, sample that has been obtained from a control subject. In a method of the present invention, a control sample may provide an average methylation status of a gene promoter of interest that is typical of a defined condition (e.g., typical of a healthy subject, or typical of a specific disease, or yet typical of glioblastoma treated according to the Stupp protocol with a given survival outcome).

The term “treatment” is used herein to characterize a method that is aimed at delaying or preventing the onset of a disease or condition (here glioblastoma); or slowing down or stopping the progression, aggravation, or deteriorations of the symptoms of the condition; or bringing about ameliorations of the symptoms of the condition; or curing the condition. In the practice of the present invention, the glioblastoma patients tested are treated according to the Stupp protocol.

The terms “approximately” and “about”, as used herein in reference to a number, generally include numbers that fall within a range of 10% in either direction of the number (higher than or lower than the number) unless otherwise stated or otherwise evident from the context (except where such number would exceed 100% of a possible value).

### DETAILED DESCRIPTION OF CERTAIN PREFERRED EMBODIMENTS

As mentioned above, the present invention provides gene promoters whose methylation status correlates with the clinical survival outcome of glioblastoma patients treated according to the Stupp protocol. Also provided are methods, and kits for using these biomarkers for the stratification of glioblastoma patients.

### I—Biomarkers

In one aspect, the present invention provides the identity of a 60 gene promoters whose methylation status is predictive of the clinical survival outcome of glioblastoma patients treated according to the Stupp protocol and/or of the response of glioblastoma patients to the Stupp treatment. These 60 promoters are promoters of the following human genes: TBX3, FSD1, FNDC3B, DGKI, AGT, FLJ25422, SEPP1, SOX10, MAP3K14, ACOT8, MGMT, KCNMB1, CHI3L2, COG4, FAM49A, GPR85, CCND1, MGC29671, LGALS1, SDPR, GPR128, NET1, SLC26A5, RNASE3, CDKN2B, NUP98, CYP24A1, ACTL6B, KLK10, TRPV4, CX36, TRIM58, GRIP1, PHLDA2, PON1, SLC2A2, TNF, FLJ23657, C1orf176, FLJ32447, HOXA11, LY6K, HMG20B, KHDRBS2, WT1, TFF2, ZNF542, ZSCAN1, ZNF540, HBZ, GPR92, HOXA9, KCNA4, RAC2, CYP1B1, FUT3, GCET2, MEGF10, GRK1, and GPX5. The full name, accession number and location of each of these genes are presented in Table 1 (see also Table 3).

TABLE 1
CpGs significantly associated with overall survival.

| GeneName | RefSeq_ID | Location | Full Name of Gene |
|---|---|---|---|
| TBX3 | NM_005996.3 | 12q24.1 | T-box 3 |
| FSD1 | NM_024333.1 | 19p 13.3 | fibronextin type III and SPRY domain containing 1 |
| FNDC3B | NM_022763.2 | 3q26.31 | fibronectin type III domain containing 3B |
| DGKI | NM_004717.2 | 7q-32.3-q33 | diacetylglycerol kinase, iota |
| AGT | NM_000029.2 | 1q42.2 | angiotensinogen (serpin peptidase inhibitor, clade |
| | | | A, member 8) |
| FLJ25422 | NM_145000.2 | 5p13.2 | RAN binding protein 3-like |
| SEPP1 | NM_005410.2 | 5q31 | selenoprotein P, plasma, 1 |
| SOX10 | NM_006941.3 | 22q13.1 | SRY (sex determining region Y)-box 10 |
| MAP3K14 | NM_003954.1 | 17q21 | mitogen-activated protein kinase kinase kinase 14 |
| SOX10 | NM_006941.3 | 22 | Transcription factor SOX-10 |
| ACOT8 | NM_183385.1 | 20q13.12 | acyl-CoA thioesterase 8 |
| MGMT | NT_008818.15 | 10 | O-6-methylguanine-DNA methyltransferase |
| KCNMB1 | NM_004137.2 | 5q34 | potassium large conductance calcium-activated |
| | | | channel, subfamily M, beta member 1 |
| CHI3L2 | NM_001025197.1 | 1p13.3 | chitinase 3-like-2 |
| COG4 | NM_015386.1 | 16q22.1 | component of oligomeric golgi complex 4 |
| FAM49A | NM_030797.1 | 2p24.2 | family with sequence similarly 49, member A |
| GPR85 | NM_018970.3 | 7q31 | G protein-coupled receptor 85 |
| CCND1 | NT_078088.3 | 11q13 | cyclin D1 |
| MGC29671 | NM_182538.3 | 17p13.2 | spinster homolog 3 (Drosophila) |
| LGALS1 | NM_002305.2 | 22q13.1 | lectin, galactoside-binding, soluble, 1 |
| SDPR | NM_004657.4 | 2q32-q33 | serum deprivation response |
| GPR128 | NM_032787.1 | 3q12.2 | G protein-coupled receptor 128 |
| NET1 | NM_005863.2 | 10p15 | neuroepithelial cell transforming 1 |
| SLC26A5 | NM_198999.1 | 7q22.1 | solute carrier family 26, member 5 (prestin) |
| RNASE3 | NM_002935.2 | 14q24-q31 | ribonuclease, RNase A family 3 |
| CDKN2B | NT_008413.17 | 9p21 | cyclin-dependent kinase inhibitor 2B (p15, inhibits |
| | | | CDK4) |
| NUP98 | NM_139131.2 | 11p15.5 | nucleoporin 98 kDa |
| CYP24A1 | NM_000782.3 | 20q13 | cytochrome P450, family 24, subfamily A, |
| | | | polypeptide 1 |
| ACTL6B | NM_016188.3 | 7q22 | actin-like 6B |
| KLK10 | NT_011109.15 | 19q13 | kallikrein-related peptidase 10 |
| TRPV4 | NM_147204.1 | 12q24.1 | transient receptor potential cation channel, |
| | | | subfamily V, member 4 |
| CX36 | NM_020660.1 | 15q14 | gap junction protein, delta 2, 36 kDa |
| TRIM58 | NM_015431.2 | 1q44 | tripartite motif containing 58 |
| GRIP1 | XM_934800.1 | 12q14.3 | glutamate receptor interacting protein 1 |
| PHLDA2 | NM_003311.3 | 11p15.4 | pleckstrin homology-like domain, family A, |
| | | | member 2 |
| PON1 | NM_000446.3 | 7q21.7 | paraoxonase 1 |
| SLC2A2 | NM_000340.1 | 3q26.1-q26.2 | solute carrier family 2 (facilitated glucose |
| | | | transporter), member 2 |
| TNF | NM_000594.2 | 6p21.3 | tumor necrosis factor |
| FLJ23657 | NM_178497.2 | 4q21.1 | chromosome 4 open reading frame 26 |
| C1orf176 | NM_022774.1 | 1p34.2 | defects in morphology 1 homolog (S. cerevisiae) |
| FLJ32447 | NM_153038.1 | 2q36.1 | coiled-coil domain containing 140 |
| HOXA11 | NM_005523.4 | 7p15.2 | homembox A11 |
| LY6K | NM_017527.2 | 8q24.3 | lymphocyte antigen 6 complex, locus K |
| HMG20B | NM_006339.1 | 19p13.3 | high mobility group 20B |
| KHDRBS2 | NM_152688.1 | 6q11.1 | KH domain containing, RNA binding, signal |
| | | | transduction associated 2 |
| WT1 | NT_009237.17 | 11p13 | Wilms tumor 1 |
| TFF2 | NM_005423.3 | 21q22.3 | trefoil factor 2 |
| ZNF542 | NM_194319.1 | 19q13.43 | zinc finger protein 542 |
| ZSCAN1 | NM_182572.2 | 19q13.43 | zinc finger and SCAN domain containing 1 |
| ZNF540 | NM_152606.2 | 19q13.12 | zinc finger protein 540 |
| HBZ | NM_005332.2 | 16p13. | hemoglobin, zeta |
| GPR92 | NM_020400.4 | 12p13.3 | lysophosphatidic acid receptor 5 |
| HOXA9 | NM_152739.2 | 7p15.2 | homeobox A9 |
| KCNA4 | NM_002233.2 | 11p14 | potassium voltage-gated channel, shaker-related |
| | | | subfamily, member 4 |
| RAC2 | NM_002872.3 | 22q13.1 | ras-related C3 botulinum toxin substrate 2 (rho |
| | | | family, small GTP binding protein Rac2) |
| CYP1B1 | NM_000104.2 | 2p22.2 | cytochrome P450, family 1, subfamily B, |
| | | | polypeptide 1 |
| FUT3 | NM_000149.1 | 19p13.3 | fucosyltransferase 3 (galactoside 3(4)-L- |
| | | | fucosyltransferase, Lewis blood group) |
| GCET2 | NM_152785.3 | 3q13.2 | germinal center expressed transcript 2 |
| MEGF10 | NM_032446.1 | 5q33 | multiple EGF-like domains |
| GRK1 | NM_002929.2 | 13q34 | G protein coupled-receptor kinase 1 |
| GPX5 | NM_001509.1 | 6p22.1 | glutathione peroxidise 5 (epididymal androgen- |
| | | | related protein) |

In particular, the Applicants have found that hypomethylation of the SOX10 promoter, in a biological sample of a glioblastoma patient, is indicative of a patient who is responsive to the Stupp treatment.

The Applicants have further found that the simultaneous hypermethylation of the MGMT promoter and one or more of: hypomethylation of the FNDC3B promoter, hypermethylation of the TBX3 promoter, hypermethylation of the DGKI promoter, hypermethylation of the FSD1 promoter, is indicative of a patient who is non-responsive to the Stupp treatment. In other words, the Applicants have found that glioblastoma patients who are predicted to be responsive to the Stupp treatment using the conventional MGMT stratification method, and who exhibit hypomethylation of the FNDC3B promoter, and/or hypermethylation of the TBX3 promoter, and/or hypermethylation of the DGKI promoter, and/or hypermethylation of the FSD1 promoter, are in fact non-responsive to the Stupp treatment.

The Applicants have also found that the methylation status of the DGKI promoter (and in particular of the DGKI_{—}7 site) allows a stratification of the MGMT-methylated patients, who are known in the art to have a better survival outcome than MGMT-unmethylated patients. In particular, they determined that while MGMT-methylated patients have an overall survival of 24.8 months, MGMT-methylated patients who exhibit hypermethylation of the DGKI promoter have an overall survival of only 16.6 months, which is not significantly different from the overall survival of MGMT-unmethylated patients (overall survival of 13.4 months). They also determined that MGMT-methylated patients who exhibit hypomethylation of the DGKI promoter have an overall survival of 29.6 months.

The Applicants have also found that the methylation status of the SDPR promoter allows a stratification of the MGMT-unmethylated patients. In particular, they determined that while MGMT-unmethylated patients have an overall survival of 13.4 months, MGMT-unmethylated patients who exhibit hypermethylation of the SDPR promoter have an overall survival of 26 months, which is close to the overall survival of MGMT-methylated patients. They also determined that MGMT-unmethylated patients who exhibit hypomethylation of the SDPR promoter have an overall survival of 10.6 months.

The Applicants have also found that the methylation status of both the DGKI promoter and SDPR promoter allows a stratification of the MGMT-methylated patients. Indeed, they determined that while MGMT-methylated patients have an overall survival of 24.8 months, MGMT-methylated patients who exhibit hypomethylation of the DGKI promoter and hypermethylation of the SDPR promoter have an overall survival of 52.3 months, which is much higher than the overall survival of MGMT-methylated patients. They also determined that MGMT-methylated patients who exhibit hypomethylation of the DGKI promoter and hypomethylation of the SDPR promoter have an overall survival of 26.3 months, which is in the same range as the overall survival of MGMT-methylated patients.

The Applicants have also found that the methylation status of both the DGKI promoter and SDPR promoter allows a stratification of the MGMT-unmethylated patients. Indeed, they determined that while MGMT-unmethylated patients have an overall survival of 13.4 months, MGMT-unmethylated patients who exhibit hypomethylation of the DGKI promoter and hypermethylation of the SDPR promoter have an overall survival of 29.7 months, which is much higher than the overall survival of MGMT-unmethylated patients. They also determined that MGMT-unmethylated patients who exhibit hypomethylation of the DGKI promoter and hypomethylation of the SDPR promoter have an overall survival of 11.7 months, which is lower than the overall survival of MGMT-unmethylated patients.

In all cases, the same stratifications were obtained based on PFS (progression-free survival) values. As used herein, the term “progression-free survival” has its art understood meaning and specifically refers herein to the length of time, during the Stupp treatment, during which glioblastoma does not get worse.

### II—Prognosis Methods

As will be appreciated by those of ordinary skill in the art, biomarkers whose methylation status correlates with survival outcome in glioblastoma patients treated according to the Stupp protocol can be used to characterize biological samples of glioblastoma patients and thereby provide a prognosis to these patients. The terms “glioblastoma”, “glioblastoma multiforme” and “GBM” are used herein interchangeably. They refer to the most common and most aggressive malignant primary brain tumor in humans, involving glial cells and accounting for 52% of all functional tissue brain tumor cases and 20% of all intracranial tumors.

Accordingly, the present invention relates to methods for predicting glioblastoma patients' response to the Stupp treatment, if these patients were to be treated according to the Stupp protocol, and/or for predicting their clinical survival outcome or progression-free survival, if these patients were to be treated according to the Stupp protocol. Based on the prognosis obtained using a prognosis method of the invention, a physician can then prescribe the best therapeutic strategy to the glioblastoma patient.

### Biological Samples

The methods provided herein may be applied to the study of any biological sample allowing the methylation status of biomarkers of the invention to be assessed. Examples of such biological samples include in particular samples of brain tissue, cerebrospinal fluid or blood, as well as cells (or their progeny) or cell content isolated from such tissues or fluids. Tissue samples may be fresh or frozen samples, or paraffin-embedded samples collected from a subject, or archival tissue samples, for example, with known diagnosis, and/or outcome history. Biological samples may be collected by any non-invasive means, such as, for example, fine needle aspiration and needle biopsy, or alternatively, by an invasive method, including for example, surgical biopsy. In certain preferred embodiments, the biological sample is a glioblastoma tissue obtained from the patient during surgery.

Preferably, the inventive methods are performed on nucleic acid extracts derived from the biological sample, in particular on genomic DNA. Genomic DNA may be obtained from DNA extracted from the brain or cerebrospinal fluid samples or from cells obtained from such samples. Methods of DNA extraction are well known in the art (see, for example, J. Sambrook et al., “Molecular Cloning: A Laboratory Manual”, 1989, 2^{nd} Ed Cold Spring Harbour Laboratory Press: New York).

A classical DNA isolation protocol is based on extraction using organic solvents such as a mixture of phenol and chloroform, followed by precipitation with ethanol. Other methods include: salting out DNA extraction (see, for example, Sunnucks et al., Genetics, 1996, 144: 747-756; and Aljanabi et al., Nucl. Acids Res. 1997, 25: 4692-4693); the trimethylammonium bromide salts DNA extraction method (see, for example, Gustincich et al., BioTechniques, 1991, 11: 298-302) and the guanidinium thiocyanate DNA extraction method (see, for example, Hammond et al., Biochemistry, 1996, 240: 298-300).

There are also numerous different and versatile kits that can be used to extract DNA from biological tissues or fluids and that are commercially available for example from BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

### Determination of Gene Promoter Methylation Status

A prognostic method according to the present invention includes a step of determining the methylation status of a biomarker (gene promoter) or a combination of biomarkers (gene promoters) of the invention in a biological sample obtained from a glioblastoma patient. Determination of the methylation status may be performed using any method known in the art to be suitable for assessing the methylation of cytosine residues in DNA. Such methods are known in the art and have been described; and one skilled in the art will known how to select the most suitable method depending on the number of samples to be tested, the quantity of sample available, and the like.

Thus, the methylation status of a gene promoter or a combination of gene promoters of the invention can be determined using any of a wide variety of methods that are generally divided into strategies based on methylation-specific PCR (MSP), and strategies employing PCR performed under methylation-independent conditions (MIP). Methylation-independent PCR (MIP) primers are used in most of the available PCR-based methods. They are designed for proportional amplification of methylated and unmethylated DNA. In contrast, methylation-specific PCR (MSP) primers are designed for the amplification of methylated template only.

Examples of methylation-independent PCR based techniques include, but are not limited to, direct bisulfite direct sequencing (Frommer et al., PNAS USA, 1992, 89: 1827-1831), pyrosequencing (Collela et al., Biotechniques, 2003, 35: 146-150; Uhlmann et al., Electrophoresis, 2002, 23: 4072-4079; Tost et al., Biotechniques, 2003, 35: 152-156), Combined Bisulfite Restriction Analysis or “COBRA” (Xiong et al., Nucleic Acids Res., 1997, 25: 2532-2534), Methylation-Sensitive Single-Nucleotide Primer Extension or “MS-SnuPE” (Gonzalgo et al., Nucleic Acids Res., 1997, 25: 2529-2531), Methylation-Sensitive Melting Curve Analysis or “MS-MSA” (Worm et al., Clin. Chem., 2001, 47: 1183-1189), Methylation-Sensitive High-Resolution Melting or “MS-HRM” (Wojdacz et al., Nucleic Acids Res., 2007, 35:e41), MALDI-TOF mass spectrometry with base-specific cleavage and primer extension (Ehrich et al., PNAS USA, 2005, 102: 15785-15790), and HeavyMethyl (Cottrell et al., Nucleic Acids Res., 2004, 32: e10).

Examples of methylation-specific PCR based techniques include for example methylation specific PCR or “MSP” (Herman et al., PNAS USA, 1996, 93: 9821-9826; Mackay et al., Hum. Genet., 2006, 120: 262-269; Mackay et al., Hum. Genet., 2005, 116: 255-261; Palmisano et al., Cancer Res., 2000, 60: 5954-5958; Voso et al., Blood, 2004, 103: 698-700), MethylLight (Eads et al., Nucleic Acids Res., 2000, 28:e32; Eads et al., Cancer Res., 1999, 59: 2302-2306; Lo et al., Cancer Res., 1999, 59: 3899-3903), Melting curve Methylation Specific PCR or “McMSP” (Akey et al., Genomics, 2002, 80: 376-384), Sensitive Melting Analysis after Real-Time MSP or “SMART-MSP” (Kristensen et al., Nucleic Acids Res., 2008, 36: e42), and Methylation-Specific Fluorescent Amplicon Generation or “MS-FLAG” (Bonanno et al., Clin. Chem., 2007, 53: 2119-2127).

A large number of these methods rely on the prior treatment of DNA with sodium bisulfite. This treatment leads to the conversion of unmethylated cytosine to uracil, while methylated cytosine remains unchanged (Clark et al., Nucleic Acids Res., 1994, 22: 2990-2997). This change in the DNA sequence following bisulfite conversion can be detected using a variety of methods, including PCR amplification followed by DNA sequencing. It is safe to say that the use of bisulfite-converted DNA for DNA-methylation analysis has surpassed almost every other methodology for DNA methylation analysis, thereby becoming the gold standard for detecting changes in DNA methylation. The protocol described by Frommer et al. (PNAS USA, 1992, 89: 1827-1831) has been widely used for sodium bisulfite treatment of DNA, and a variety of commercial kits are now available for this purpose.

Thus, in a prognostic method according to the invention, the step of determining the methylation status of a gene promoter, or of a combination of gene promoters of the invention, may be performed using any of the techniques described above or any combination of these techniques. One skilled in the art will recognized that when the methylation status of a combination of gene promoters has to be determined, the determinations may be performed using the same DNA methylation analysis technique or different DNA methylation analysis techniques.

### Glioblastoma Prognosis

Once the methylation status of the gene promoter or combination of gene promoters has been determined (e.g., as described above) for the biological sample being tested, it may be compared to the methylation status of the same gene promoter(s) in one or more control samples or reference samples. As already mentioned above, the term “methylation status” encompasses the relative abundance (e.g., percentage) of methylated cytosine residues of CpG sites in a gene promoter as well as the presence or absence of such methylated cytosine residues. When the methylation status is determined as a relative abundance, comparison will preferably be performed after correction for differences in the amount of sample assayed and the variability in the quality of the sample used (e.g., amount and quality of genomic DNA tested). Correction may be carried out using any suitable method well-known in the art. Control or reference samples may be obtained from healthy individuals (i.e., from normeoplastic brains) and/or from individuals afflicted with glioblastoma and treated according to the Stupp protocol and whose survival outcome is known.

In addition or alternatively, once the methylation status of the gene promoter or combination of gene promoters of the invention has been determined for the biological sample being tested, it may be compared to the methylation status of the same gene promoter(s) as determined by the present Applicants. Thus, the methylation status determined for the biological sample tested may be compared to the DNA methylation data that the present Applicants have submitted to the Gene Expression Omnibus (GEO) repository under accession number “GSE22867”. Alternatively, the methylation status determined for the biological tested may be compared to the threshold values provided in the Examples section below.

Based on the results of the comparison, a prognosis may be provided. The term “providing a prognosis for a glioblastoma patient” is used herein to mean predicting the patient's response to a Stupp treatment, if said patient were to be treated according to the Stupp protocol. Thus, in certain embodiments, providing a prognosis includes providing the expected overall survival outcome for the patient, if said patient were to be treated according to the Stupp protocol. In other embodiments, providing a prognosis includes providing the expected progression-free survival for the patient, if said patient were to be treated according to the Stupp protocol. In yet other embodiments, providing a prognosis includes indicating whether or not the patient would be responsive to the Stupp treatment, if said patient were to be treated according to the Stupp protocol. As one skilled in the art will recognize, providing a prognosis may include providing both the progression-free survival and overall survival.

Thus, as demonstrated by the present Applicants, hypomethylation of the SOX10 promoter is indicative of a glioblastoma patient who would be responsive to the Stupp treatment, if said patient were to be treated according to the Stupp protocol.

In MGMT-methylated glioblastoma patients, hypomethylation of the FNDC3B promoter, and/or hypermethylation of the TBX3 promoter, and/or hypermethylation of the DGKI promoter, and/or hypermethylaltion of the FSD1 promoter, is/are indicative of a patient who would be non-responsive to the Stupp treatment, if said patient were to be treated according to the Stupp protocol.

In MGMT-methylated glioblastoma patients, hypermethylation of the DGKI promoter is indicative of a short-term survival outcome and of a patient who would be non-responsive to the Stupp treatment, if said patient were to be treated according to the Stupp protocol.

As used herein, the term “short-term survival outcome” refers to an overall survival that is shorter than the overall survival determined by the standard MGMT promoter methylation status method for a MGMT-methylated glioblastoma patient and/or to a progression-free survival that is shorter than the progression-free survival determined by the standard MGMT promoter methylation status method for a MGMT-methylated glioblastoma patient. The present Applicants have determined that the median value of the short-term overall survival is 13.7 months, and that the median value of the short-term progression-free survival is 8.9 months.

In MGMT-unmethylated glioblastoma patients, hypomethylation of the SDPR promoter is indicative of a patient who would be non-responsive to the Stupp treatment and of a short-term survival outcome (see above), if said patient were to be treated according to the Stupp protocol.

In MGMT-methylated glioblastoma patients, hypomethylation of the DGKI promoter and hypermethylation of the SDPR promoter is indicative of a patient who would be responsive to the Stupp treatment and of a long-term survival outcome, if said patient were to be treated according to the Stupp protocol.

As used herein, the term “long-term survival outcome” refers to an overall survival that is longer than the overall survival determined by the standard MGMT promoter methylation status method for a MGMT-methylated glioblastoma patient and/or to a progression-free survival that is longer than the progression-free survival determined by the standard MGMT promoter methylation status method for a MGMT-methylated glioblastoma patient. The present Applicants have determined that the median value of the long-term overall survival is 52.3 months, and that the median value of the long-term progression-free survival is 22.7 months.

In MGMT-methylated glioblastoma patients, hypomethylation of the DGKI promoter and hypomethylation of the SDPR promoter is indicative of a medium-term survival outcome, if the patient were to be treated according to the Stupp protocol.

As used herein, the term “medium-term survival outcome” refers to an overall survival that is close to the overall survival determined by the standard MGMT promoter methylation status method for a MGMT-methylated glioblastoma patient and/or to a progression-free survival that is longer than the progression-free survival determined by the standard MGMT promoter methylation status method for a MGMT-methylated glioblastoma patient. The present Applicants have determined that the median value of the medium-term survival is 26 months, and that the median value of the medium-term progression-free survival is 14.2 months.

In MGMT-unmethylated glioblastoma patients, hypermethylation of the SDPR promoter is indicative of a medium-term survival outcome (see above) for the patient, if the patient were to be treated according to the Stupp protocol.

### Selection of Appropriate Treatment

Based on the prognosis obtained using a method of the present invention, skilled physicians may select and prescribe treatments adapted to the patients. Thus, to glioblastoma patients who are predicted to be responsive to the Stupp treatment or to have a medium-term survival outcome or long-term survival outcome, were they to be treated according to the Stupp protocol, a physician will prescribe the Stupp treatment. In contrast, to glioblastoma patients who are predicted to be unresponsive to the Stupp treatment or to have a short-term survival outcome, were they to be treated according to the Stupp protocol, a physician will not prescribe the Stupp treatment, but an alternative treatment or will direct said patients to glioblastoma clinical trials. Thus, prognostic methods according to the invention may be used for patient selection in glioblastoma clinical trials.

### III—Kits

In another aspect, the present invention provides kits comprising materials useful for carrying out the prognosis methods of the invention. The prognosis procedures described herein may be performed by diagnostic laboratories, experimental laboratories, and practitioners.

Materials and reagents for characterizing biological samples from glioblastoma patients may be assembled together in a kit. In certain embodiments, an inventive kit comprises primers for the amplification of regions of bisulfite-treated genomic DNA that contain the CpG sites of interest, in particular the CpG sites contained in a gene promoter of the invention or in a combination of gene promoters of the invention. As indicated above, the primers may be methylation-independent PCR (MIP) primers or methylation-dependent PCR (MSP) primers. A kit of the invention may comprise both MIP primers and MSP primers. One skilled in the art will know how to design MIP and/or MSP primers for the gene promoters of the invention (Dahiya, Bioinformatics, 2002, 18: 1427-1431; Barekati et al., Obstet. Gynecol. Int., 2010: 870865; Li, Methods Mol. Biol., 2007, 402: 37-384; Pattyn et al., BMC Bioinformatics, 2006, 7: 496).

In other embodiments, an inventive kit comprises pyrosequencing primers allowing determination of the methylation status of a gene promoter or a combination of gene promoters of the invention by pyrosequencing. One skilled in the art will know how to design pyrosequencing primers for the gene promoters of the invention (Dejeux et al., Methods Mol. Biol., 2009, 507: 189-205; Guo et al., Methods Mol. Biol., 2007, 373: 57-62; Barkensiek et al., Clin. Chem., 2007, 53: 17-23).

A kit may further comprise instructions for using the kit according to a method of the invention. Depending on the procedure, the kit may further comprise one or more of: extraction buffer and/or reagents (in particular genomic DNA extraction), buffer and/or reagents for bisulfite treatment of genomic DNA, amplification buffer and/or reagents, hybridization buffer and/or reagents, labeling buffer and/or reagents, and detection means. Protocols for using these buffers and reagents to perform different steps of the procedure may be included in the kit.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

Instructions for using the kit according to a method of the invention may comprise instructions for processing the biological sample obtained from the glioblastoma patient, and/or instructions for bisulfite-converting genomic DNA, and/or instructions for performing the test, and/or instructions for interpreting the results as well as a notice in the form prescribed by a governmental agency (e.g., FDA) regulating the manufacture, use or sale of pharmaceuticals or biological products.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results reported below were described in two scientific papers (A. Etcheverry et al., “DNA methylation in glioblastoma: impact on gene expression and clinical outcome”, BMC Genomics, Dec. 14, 2010, 11: 701; and A. Etcheverry et al., “DGKI and SDPR methylation status modulate the prognostic value of the MGMT promoter methylation status in glioblastoma patients treated with combined radio-chemotherapy”, submitted for review late July 2011). The content of each of these scientific papers is incorporated herein by reference in its entirety, including the supplemental information and figures.

### Example 1

### DNA Methylation in Glioblastoma: Impact on Gene Expression and Clinical Outcome

### Patients and Methods

### Tissue Samples.

The prospective cohort included 55 patients with newly diagnosed glioblastoma (World Health Organization (WHO) grade IV), admitted to the Neurosurgery Departments of Rennes and Angers University Hospitals. Tumor samples were collected, following informed consent, in accordance with the French regulations and the Helsinki Declaration. Initial histologic findings were confirmed, according to the WHO classification (Louis et al., Acta Neuropathol, 2007, 114: 97-109) by a central review panel including at least two neuropathologists. The male/female ratio was 1:0.96. Median age at diagnosis was 57.5±12 years (range: 26-80 years) and median preoperative Karnofsky Performance Status (KPS) was 78.6 (range: 40-100). Fifty patients underwent radiotherapy and chemotherapy with concomitant and adjuvant temozolomide (Stupp protocol) following surgery. Four patients received only fractionated radiotherapy (60 Gy). One patient died after surgery. Median overall survival (OS) was 18.7±17.3 months (range: 0.2-98.6 months). Five non-neoplastic brain tissues obtained from patients undergoing surgery for chronic epilepsy were included in the study as control samples. Each snap-frozen tumor block was cut into 10 μm sections. For accurate paired comparisons between biological materials, adjacent sections were used for DNA and RNA extraction. The expression profiles of 40 glioblastomas for which methylation data were also available were investigated.

### DNA and RNA Isolation.

DNA was extracted using the NucleoSpin Tissue Kit (Macherey Nagel) according to the manufacturer's instructions. The quality of DNA samples was assessed by electrophoresis in a 1% agarose gel. Total RNA was isolated with the NucleoSpin RNAII Kit (Macherey-Nagel). RNA integrity (RNA Integrity Number ≧8) was confirmed with using the Agilent 2100 Bioanalyzer (Agilent Technologies).

### DNA Methylation Profiling.

DNA methylation profiling was performed with the Infinium HumanMethylation27 beadchip (Illumina Inc.), which interrogates 27,578 highly informative CpG sites located within the proximal promoter regions of 14,475 genes (1,126 cancer-related genes). Nearly 73% of these CpGs were localized within CpG islands. DNA from glioblastomas and control brains were bisulfite-modified, using the EZ DNA methylation kit (Zymo Research) and hybridized according to the manufacturer's instructions. The profiling was performed on 55 glioblastomas and 3 normeoplastic brains. Two intra- and inter-array replicates, the first one on a glioblastoma sample and the other one on a non-neoplastic brain sample, were performed. The observed correlations between replicate samples (r>0.99) demonstrated the high reproducibility of the technique. For each interrogated CpG site, methylation status was calculated by dividing the signal from the methylated probe (M) by the sum of signals for both methylated and unmethylated (U) probes (GenomeStudio 2008.1, Illumina Inc.): β=Max(M,0)/[Max(M,0)+Max(U,0)+100]. This β-value provides a continuous and quantitative measurement of DNA methylation, ranging from 0 (completely unmethylated) to 1 (completely methylated). Missing values were imputed by nearest neighbor averaging (impute R package). DNA methylation values followed a non symmetric bimodal distribution and CpG sites were globally hypomethylated in both glioblastoma and control brain samples (median β-value=0.1). DNA methylation data have been submitted to Gene Expression Omnibus (GEO) repository under accession number “GSE22867”.

Determination of Methylation Thresholds on the Basis of Expression Value. CpG probes were binned according to their β-values (windows 0.05 wide). For each bin, the maximum expression values of the genes corresponding to the CpG probes were averaged for all patients (n=40).

### Differentially Methylated (DM) CpG Sites.

Prior to selection of the CpG sites displaying the highest DNA methylation, a variation was carried out based on the standard deviation (SD≧0.1). β-values were compared between glioblastomas and control brain tissues with Student t-tests with a Welch approximation. Adjusted p-values were calculated by controlling for the false discovery rate (FDR) using the Benjamini & Hochberg (BH) procedure (multtest, R package). CpG sites were considered significantly differentially methylated if the adjusted p-value was below 0.01 and the difference in β-values (Δβ GBM vs. control brain) was greater than 0.2.

### Pyrosequencing Analysis.

MGMT promoter pyrosequencing was performed with the PyroMark Q96 CpG MGMT kit (Qiagen), according to the manufacturer's protocol. The values obtained were averaged over the five CpG loci tested.

### Gene Expression Profiling.

The gene expression profiling study was performed using 40 glioblastoma samples and 3 non-neoplastic brain samples as controls. Gene expression profiling was carried out using the Agilent Whole Human Genome 4×44 K Microarray Kit (Agilent Technologies). Total RNA was extracted, labelled and hybridized according to the kit manufacturer's recommendations. Data were log 2-transformed and normalized (quantile normalization and baseline transformation) with Gene-Spring GX software (Agilent Technologies). Gene expression data have been submitted to Gene Expression Omnibus (GEO) repository under accession number “GSE22866”.

### Differentially Expressed (DE) Genes.

A non-parametric rank product method was used to account for hybridization bias and to identify genes that are up- or down-regulated in glioblastomas vs. control brains (RankProd R package). Genes were considered significantly differentially expressed if the FDR was below 0.05 and the absolute fold-change (GBM vs. control brain) was greater than 2.

### Correlation Analysis.

The correlation analysis was performed on 40 glioblastoma samples for which methylation and expression data were available. Methylation and expression probes were paired on the basis of Entrez Gene ID concordance. The association between CpG site methylation and the level of expression of the corresponding genes was assessed by calculating Pearson's correlation coefficient (r). The level of gene expression was considered to be inversely correlated with CpG site methylation level if the r value obtained was less than −0.5 and the p-value was less than 0.001.

### Survival Analysis.

Survival analyses were carried out on 50 patients who had undergone surgery, radiotherapy, and chemotherapy with concomitant and adjuvant temozolomide (the Stupp protocol). Univariate Cox regression analyses were performed on the CpG sites displaying the greatest variation of DNA methylation (SD>0.15). β-values were used as the predictor and OS time (in months) was used as the response. CpG sites with a p-value lower than 0.05 were selected for further analysis. For each CpG site, the β-value threshold giving the best stratification p-value according to the log-rank test was selected for the identification of patients displaying hypomethylation (β-value ≦threshold) and hypermethylation (β-value >threshold). Only CpG sites with a p-value below 0.001 were investigated further. Survival probabilities at 18 months, corresponding to the median OS in the present cohort, were determined with a classical Cox model. Time-dependent ROC curve analyses were used to determine the area under the curve (AUC) for each CpG. All tests were stratified for the age of patients (above or below the age of 50 years). Analyses were carried out with the survival and survivalROC packages of R software.

### IDH1 Mutation.

The genomic region spanning wild-type R132 of IDH1 was analyzed by direct sequencing as previously described (Kang et al., Int J Cancer, 2009, 125: 353-355).

### Results

Selection of CpG Probes with Direct Effect on Gene Expression.

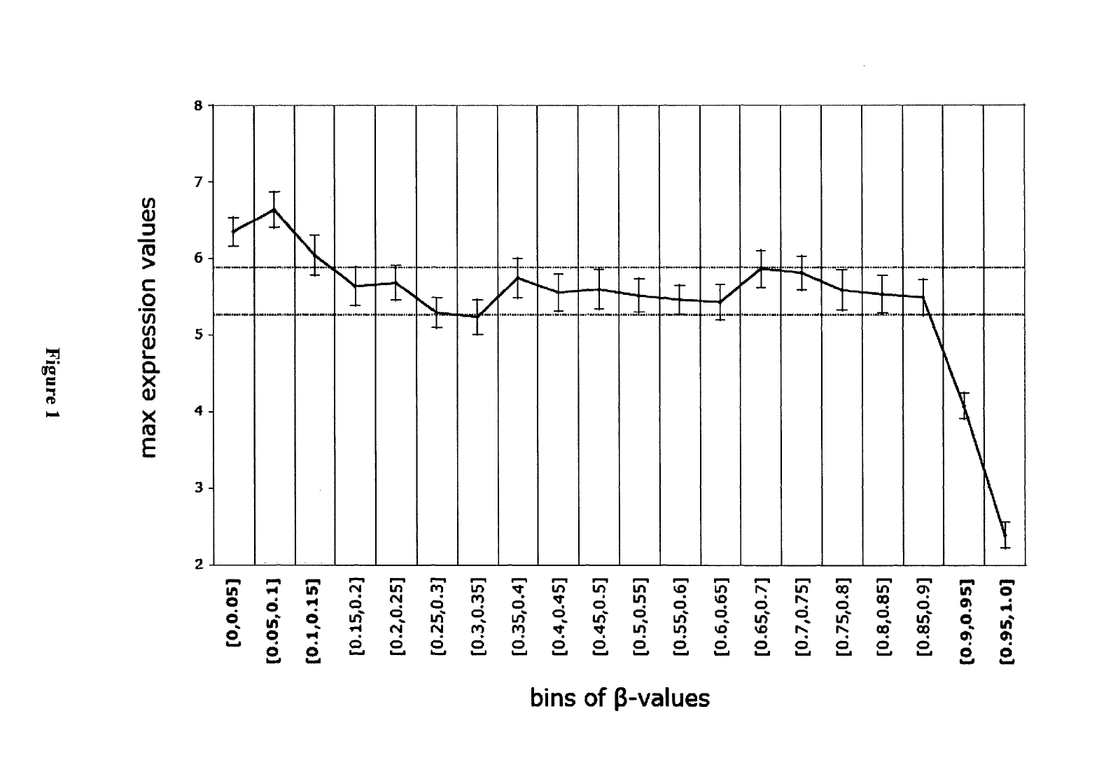

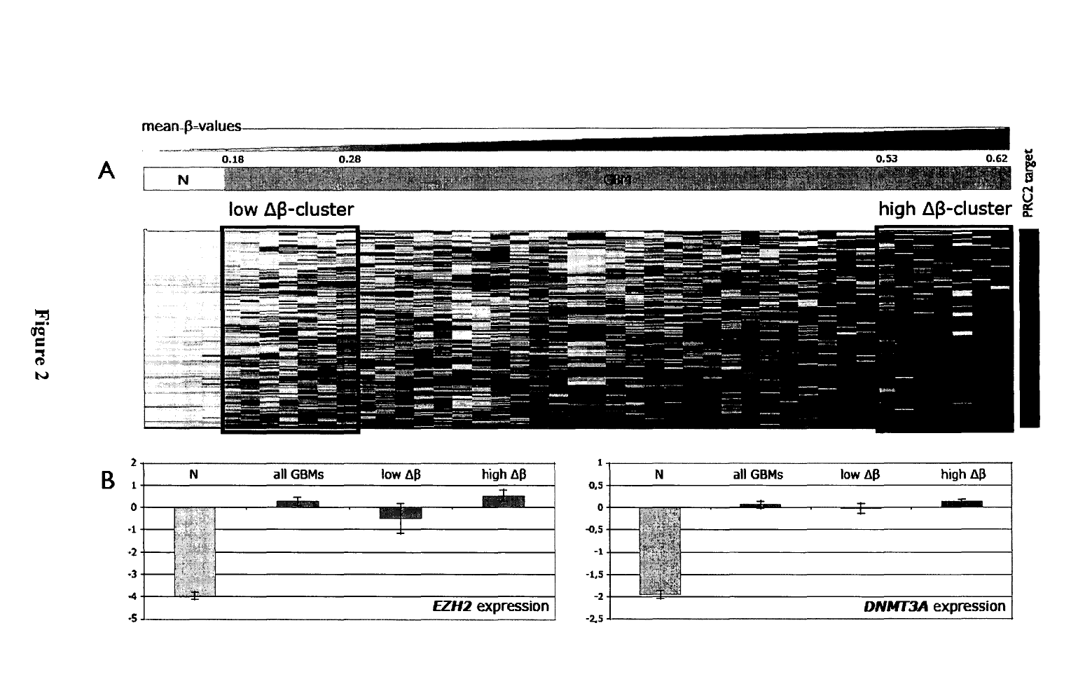

Expression levels remained almost constant for a broad range of β-values but the distributions were different for extremely low and high methylation values (FIG. 1). CpG sites with a putative effect on gene expression levels were identified as those with β-values below 0.15 or above 0.9 in at least three samples. This selection method led to the identification of 19,837 CpG sites (located within the promoter of 11,855 genes) and was used for DNA methylation profiling and correlation analysis.

### DNA Methylation Profiling of Glioblastomas.

616 of the 4,344 selected CpG sites (SD≧0.10) were found to be differentially methylated (DM) between glioblastoma and control brain samples: 440 CpG sites (358 genes) were hypermethylated and 176 (170) were hypomethylated in glioblastoma. Some of the identified changes in gene methylation have been reported before: the hypermethylation of CDKN2A (p14ARF and p161NK4a) has been implicated in carcinogenesis and tumor progression (Costello et al., Cancer Res, 1996, 56: 2405-2410), whereas the hypomethylation of S100A2 (Martinez et al., Epigenetics 2007, 2: 147-150) has been identified as a strong inducer of metastasis in vivo in non small cell lung cancer (Bulk et al., Clin Cancer Res, 2009, 15: 22-29). As expected, unsupervised hierarchical clustering of the DM CpG sites clustered the samples into two distinct groups: the glioblastoma samples and the control brain samples. CpG sites methylation patterns differed considerably between glioblastoma patients. This heterogeneity was even more marked if the hypermethylated CpG subset was considered. This analysis also showed that some glioblastoma samples were more strongly altered than others and three main glioblastoma clusters displaying different degrees of DNA methylation alteration were observed.

Functional annotation of the DM genes (NIH-DAVID software) identified several enriched Gene Ontology (GO) biological processes (Fisher Exact test). Hypermethylated genes were significantly associated with nervous system development (p-value=7×10⁻¹⁵), embryonic development (p-value=3×10⁻¹³), brain development (p-value=6×10⁻¹⁶), and cell migration (p-value=4×10⁻⁴). Hypomethylated genes were significantly associated with immune response (p-value=) 1×10⁻¹⁰) and response to stress (p-value=8×10⁻¹⁶).

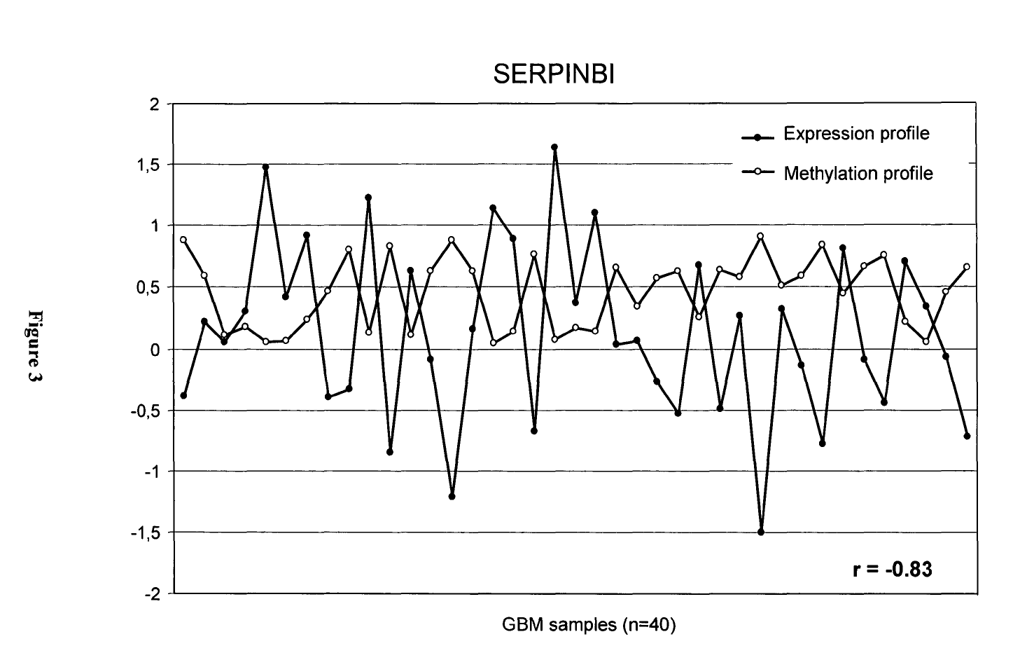

Interestingly, 97% of the hypermethylated CpG sites were located within a CpG island, whereas 91% of the abnormally demethylated CpG sites were not located within a CpG island. The frequencies of PRC2 marks were compared in the hypermethylated gene set and in the full array, as previously described by Martinez et al. (Epigenetics, 2009, 4: 255-264). The hypermethylated gene set was significantly enriched in PRC2 targets (35% vs. 9.5%, Fisher's exact test p-value=2×10⁻¹⁶). This suggests that a large proportion of the hypermethylated genes in glioblastoma may have undergone de novo DNA methylation mediated by the PRC2 complex. This hypothesis was tested by carrying out unsupervised hierarchical clustering restricted to the hypermethylated CpGs located within PRC2-targeted promoters (FIG. 2A). Considerable heterogeneity was observed between glioblastomas, and the present applicants focused on two groups of seven patients clustered on the basis of the difference between their mean β-value and that of control brain (4). These groups are named the “low-Δβ” (mean Δβ=0.15) and “high-Δβ” (mean Δβ=0.49) groups. The expression levels of genes belonging to the PRC2 complex (EZH2, SUZ12, EED) and of DNMT genes (DNMT1, DNMT3A and DNMT3B) were compared in control brains, all the glioblastoma samples, the low-Δβ cluster and the high-Δβ cluster. Two genes (EZH2 and DNMT3A) were significantly over-expressed in glioblastomas relative to control brains (FDR=0, foldchange=19 and FDR=0.003, fold-change=4, respectively). These two genes were more strongly expressed in the high-Δβ cluster, but no statistically significant difference was found between the levels of expression in the low- and high-Δβ clusters (FIG. 2B).

### Correlation Analysis.

In total, 421 CpG sites (321 genes) displayed a significant inverse correlation (r<−0.5) between methylation level and the level of expression of the corresponding gene in glioblastoma samples. Almost 91% of these sites were located within CpG islands. The genes displaying the strongest inverse correlation included four genes related to cancer processes: SERPINB1 (FIG. 3), which promotes cancer cell motility in invasive oral squamous cell carcinoma (Tseng et al., Oral Oncol, 2009, 45: 771-776), EMP3, which displays regulation through promoter methylation in gliomas (Alaminos et al., Cancer Res, 2005, 65: 2565-2571), FABP5, which mediates EGFR-induced carcinoma cell growth (Kannan-Thulasiraman et al., J Biol Chem, 2010, 285: 19106-19115), and CBR1, which is involved in tumor progression (Miura et al., Mol Cell Biochem, 2008, 315: 113-121; Takenaka et al., Cancer Epidemiol Biomarkers Prev, 2005, 14: 1972-1975). Thirteen genes were differentially expressed in glioblastoma vs. control brain, consistent with their promoter methylation status (5 overexpressed genes with a hypomethylated promoter: B3GNT5, FABP7, ZNF217, BST2 and OAS1; 8 underexpressed genes with a hypermethylated promoter: SLC13A5, GSTM5, ME1, UBXD3, TSPYL5, FAAH, C7orf13, and C3orf14).

### Survival Analysis.

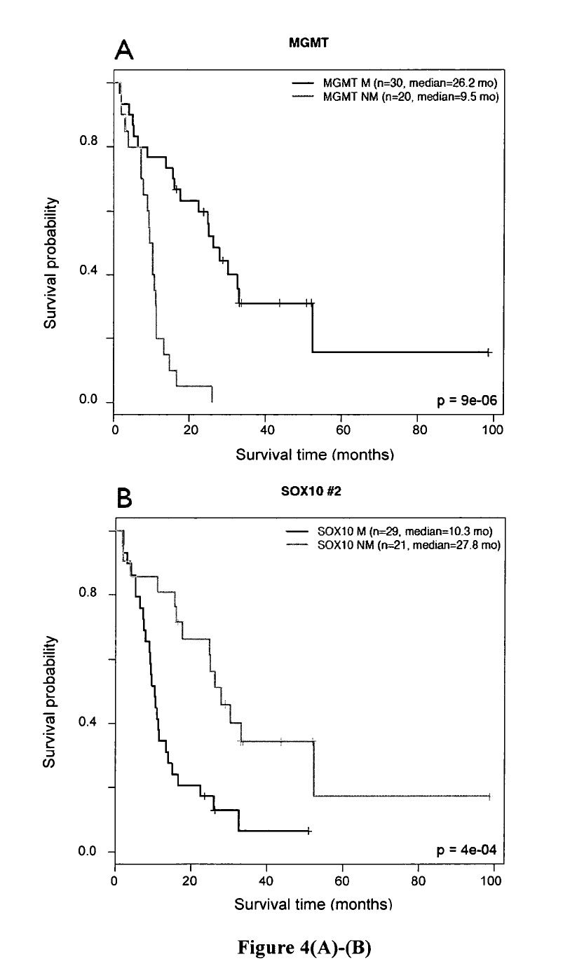

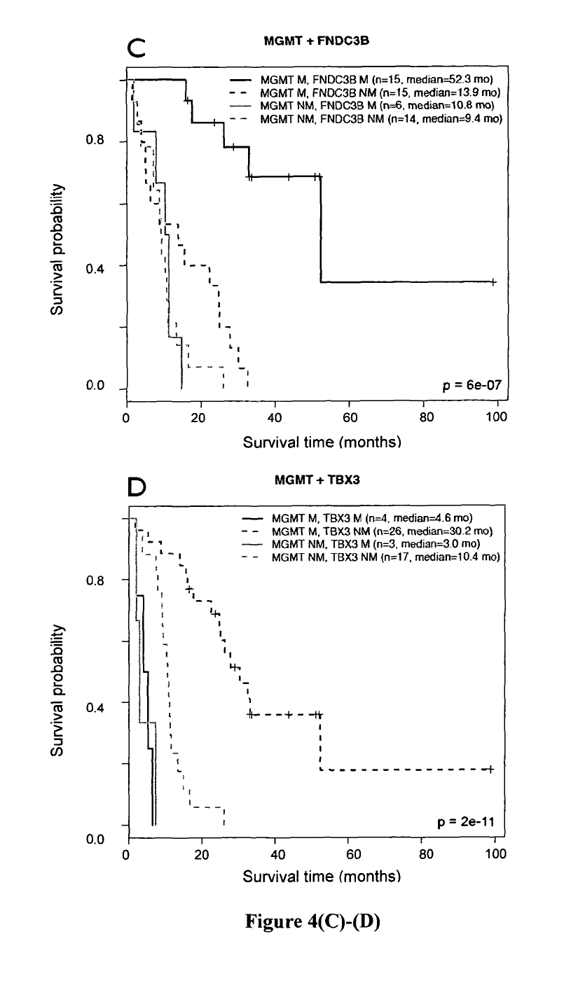

Univariate Cox analyses identified 474 CpG sites (419 genes) significantly associated with overall survival (OS). These sites had a high predictive power (absolute univariate z score greater than 2) and 26 were inversely correlated. As expected, the methylation status of the five CpG sites located within the MGMT promoter was correlated with survival. Sixty CpG sites stratified the patients into two groups (each containing at least five patients) with significantly different OS (FIG. 4).

TABLE 2
Survival analysis for 50 GBM patients treated with the Stupp protocol.

| | Univariate Cox Regression | | | Log Rank Test | | Multivariate Cox Regression | | |
|---|---|---|---|---|---|---|---|---|
| Variable | HR | 95% CI | p-value | β cut-off | p-value | HR | 95% CI | p-value |
| Age (≧50 yr vs. | 1.8 | 0.9-3.9 | 0.1 | — | — | — | — | — |
| <50 yr) | | | | | | | | |
| Sex (male vs. | 1.5 | 0.8-2.8 | 0.3 | — | — | — | — | — |
| female) | | | | | | | | |
| KPS (≧80 vs. | 1.2 | 0.6-2.3 | 0.6 | — | — | — | — | — |
| <80) | | | | | | | | |
| TBX3 | 113 | 15.1-851.9 | 4 · 10⁻⁶ | 0.45 | 1 · 10⁻⁸ | 0.05 | 0.01-0.2 | 3 · 10⁻⁵(*) |
| FSD1 | 18 | 2.9-112 | 0.002 | 0.70 | 3 · 10⁻⁷ | 0.2 | 0.09-0.6 | 0.002(*) |
| FNDC3B | 0.08 | 0.01-0.4 | 0.002 | 0.55 | 7 · 10⁻⁵ | 3.1 | 1.4-6.9 | 0.005 |
| DGKI | 77 | 9.5-616.5 | 4.10⁻⁵ | 0.45 | 3 · 10⁻⁶ | 0.3 | 0.1-0.7 | 0.008 |
| FLJ25422 | 0.05 | 0.007-0.3 | 0.002 | 0.70 | 4 · 10⁻⁴ | 2.9 | 1.2-7.2 | 0.02 |
| SEPP1 | 0.008 | 0.003-0.2 | 0.004 | 0.10 | 2 · 10⁻⁴ | 2.3 | 1.1-4.7 | 0.02 |
| SOX10 # 1 | 10 | 1.6-67.2 | 0.01 | 0.70 | 1 · 10⁻⁴ | 0.4 | 0.2-0.9 | 0.03 |
| CCND1 | 31 | 4.3-216 | 6 · 10⁻⁴ | 0.75 | 2 · 10⁻⁴ | 2.3 | 0.9-5.5 | 0.1 |
| SOX10 # 2 | 12 | 1.9-74.8 | 0.008 | 0.80 | 4 · 10⁻⁴ | 0.5 | 0.2-1.2 | 0.1 |
| ZNFN1A3 | 0.11 | 0.02-63.8 | 0.02 | 0.35 | 9 · 10⁻⁴ | 1.8 | 0.8-4.2 | 0.2 |
| MGMT | 0.18 | 0.04-0.8 | 0.02 | 0.10 | 9 · 10⁻⁶ | — | — | — |

Log rank tests were performed between methylated and non-methylated patients.
The multivariate analysis includes the methylation status of the tested CpG site and MGMT.
MGMT methylation status was always significantly associated with OS (p-value < 0.05) and
(*)indicates which CpG site had a lower p-value than the one observed for MGMT in the multivariate model.
(yr = year; HR = Hazard Ratio; CI = Confidence Interval).

One of these sites is located within the MGMT promoter (Table 2 and FIG. 4A) and its Illumina probe overlaps the sequence tested by the PyroMark Q96 CpG MGMT kit used to validate the present data. For this CpG site, a strong correlation was obtained between the results of the two techniques (r=0.7). Interestingly, 10 CpG sites (9 genes) had a larger AUC than the MGMT CpG (Kruskal-Wallis test p-value <5×10⁻⁴) (Table 2). For these 10 CpGs no evidence of violation of the proportional hazards assumption was found. The hypermethylation of two of these CpG sites, within the SOX10 promoter, was associated with shorter survival (FIG. 4B). CpG site #2 methylation level was inversely correlated with the level of SOX10 expression (r=−0.75) in glioblastoma samples, and SOX10 was significantly underexpressed in glioblastoma (FDR=0.009, fold-change=4). This inverse correlation and underexpression in glioblastoma, is entirely consistent with the shorter survival observed for patients displaying SOX10 hypermethylation. Four CpG sites remained significantly associated with OS (p-value <0.01) in a Cox multivariate model including MGMT promoter methylation status and were therefore identified as potential independent prognostic markers. These sites are located within the promoters of the FNDC3B, TBX3, FSD1, and DGKI genes (FIGS. 4C and D).

### Discussion

Array technology was used in this study for quantitative expression and methylation profiling in a well characterized cohort of newly diagnosed glioblastoma patients. The applicants provide (i) a relationship between DNA methylation pattern and gene expression in glioblastoma and (ii) a relationship between DNA methylation and clinical outcome in a subgroup of patients given uniform treatment in accordance with the STUPP protocol.

The methylation analysis identified 616 CpG sites differentially methylated between glioblastoma and control brain and revealed considerable heterogeneity between glioblastomas, particularly for hypermethylated CpG sites. Hypo- and hypermethylated CpG sites were preferentially located outside and within CpG islands, respectively. This clearly confirms that cancer cells are characterized by both a loss of methylation in CpG-depleted regions and gains of methylation at CpG islands (Herman et al., N Engl J Med, 2003, 349: 2042-2054). Consistent with the findings of Martinez et al. (Martinez et al., Epigenetics, 2009, 4: 255-264), the hypermethylated gene set was found to be significantly enriched in PRC2 targets, highlighting the putative role of polycomb group proteins in de novo methylation in glioblastoma. However, the present data were not entirely consistent with this hypothesis. Indeed, there is no strong methylation pattern among the PRC2 targeted promoters and the changes in expression of the PRC2 and DNMT genes do not follow the hypermethylation gradient observed between low- and high-Δβ GBM clusters. This suggests that other genes may be linked to polycomb-associated de novo methylation.

The integrated analysis of DNA methylation and gene expression showed that DNA methylation only partly regulated gene expression. Indeed, almost a quarter of the differentially methylated genes also displayed concordant differential expression (chi-square test p-value <0.01) and, in glioblastoma samples, only 3% of the genes displayed an inverse correlation between promoter methylation and expression levels. This finding is consistent with published data for glioblastoma (Noushmehr et al., Cancer Cell, 2010, 17: 510-522). Moreover, many other well known mechanisms are involved in the regulation of gene expression (e.g., copy number alterations—de Tayrac et al., Genes Chromosomes Cancer, 2009, 48: 55-68; Nigro et al., Cancer Res, 2005, 65: 1678-1686—transcription factor production and recruitment, histone modifications, micro-RNA expression—Nagarajan et al., Semin Cancer Biol, 2009, 19: 188-197). Nevertheless, the present analysis led to the identification of 13 genes displaying concordant differential methylation and differential expression in glioblastoma and control brain, and whose methylation and expression patterns were anticorrelated. The expression patterns of these genes may therefore be tightly regulated by epigenetic mechanisms, and their in-depth analysis may help us understand the contribution of DNA methylation to glioblastomagenesis. Most of these genes have already been implicated in cancer-related processes. For example, ZNF217 (encoding zinc finger protein 217) is an important oncogene in many cancer types and its overexpression has been implicated in cell immortalization and resistance to chemotherapy (Quinlan et al., Biochim Biophys Acta, 2007, 1775: 333-340). A recent study demonstrated that the ZNF217 protein forms nuclear complexes with several histone-modifying proteins (including EZH2) with synergistic effects in transcriptional repression (Banck et al., Epigenetics, 2009, 4: 100-106). Another example is provided by FABP7 (brain fatty acid binding protein 7), which is expressed by the radial glia and involved in glia-guided neuronal migration (Feng et al., Neuron, 1994, 12: 895-908). This protein has been associated with pure glioblastoma histology, invasion and poor prognosis (Kaloshi et al., J Neurooncol, 2007, 84: 245-248). Yet another example is provided by TSPYL5 (encoding testis-specific Y-like protein), which is a potent tumor suppressor gene and a frequent target of epigenetic silencing in glial tumors and gastric cancers (Kim et al., Cancer Res, 2006, 66: 7490-7501; Jung et al., Lab Invest, 2008, 88: 153-160). This gene has been shown to play a role in cell growth and resistance to radiation, through regulation of the p21 (WAF1/Cip1) and PTEN/AKT pathway (Kim et al., Biochem Biophys Res Commun, 2010, 392: 448-453).

Noushmehr et al. (Cancer Cell, 2010, 17: 510-522) described a rare subgroup of glioblastomas with a CpG Island Methylator Phenotype. These G-CIMP tumors are a subclass of the GBM proneural subtype defined by Phillips et al. (Cancer Cell, 2006, 9: 157-173) and Verhaak et al. (Cancer Cell, 2010, 17: 98-110). They were shown to be associated with secondary and recurrent glioblastomas, IDH1 somatic mutation, younger age at diagnosis and longer survival. Using the G-CIMP 8-gene signature that these authors described (ANKRD43, HFE, MAL, LGALS3, FAS-1, FAS-2, RHO-F, and DOCK5), the present applicants identified 3 G-CIMP-positive tumors in the 55 patients of the cohort used. This proportion (5.5%) is similar to that reported in the context of the TCGA (7.6%). The association of G-CIMP status with IDH1 somatic mutation (Fisher's exact test p-value=2e-4) and younger age at diagnosis (Wilcoxon rank sum test p-value=0.01) was also confirmed. However, the applicants were unable to test the association between G-CIMP-positive status and OS, due the low frequency of this phenotype (three patients, of which only two have available survival data).

Survival analysis was performed on a cohort of 50 patients uniformly treated by the Stupp protocol (Stupp et al., N Engl J Med, 2005, 352: 987-996). To the applicants' knowledge, this is the largest uniformly treated GBM cohort ever to be studied over such a large number of CpG loci. Table 3 shows the CpGs that were found to be significantly associated with overall survival.

As expected, MGMT promoter methylation was strongly associated with longer survival, in both the microarray and pyrosequencing approaches. The chosen cutoff point for the β-value (10%) is similar to frequently used values (9%) (Dunn et al., Br J Cancer, 2009, 101: 124-131). For the 27,578 CpG sites tested, MGMT methylation status remained one of the most powerful predictors of response to temozolomide-based treatment in glioblastoma. Nevertheless, the applicants have also identified two different types of prognostic markers. The first type stratifies the patients similarly to MGMT, but with a higher AUC. There is an association between the methylation level of MGMT and SOX10 promoters (chi-square test p-value <0.01). The SOX10 gene is one such marker, and the hypermethylation of its promoter was associated with shorter survival in the present cohort. Interestingly, the SOX10 protein is a marker of oligodendrocytes (Stolt et al., Genes Dev, 2002, 16: 165-170), and the presence of oligodendroglial differentiation areas in glioblastoma has also been associated with longer survival (Salvati et al., J Neurooncol, 2009, 94: 129-134). The second type of prognostic marker (FNDC3B, TBX3, DGKI, and FSD1) identifies patients with MGMT methylated tumors not responding to STUPP treatment.

TABLE 3
CpGs significantly associated with overall survival.

| | | | | optimum | | |
|---|---|---|---|---|---|---|
| GeneName | RefSeq_ID | C No. | MapInfo | β cut-off | p-value | AUC |
| TBX3 | NM_005996.3 | 12 | 113606437 | 0.45 | 2.01E−08 | 0.724 |
| FSD1 | NM_024333.1 | 19 | 4256090 | 0.7 | 9.12E−08 | 0.773 |
| FNDC3B | NM_022763.2 | 3 | 173312843 | 0.55 | 4.87E−05 | 0.737 |
| DGKI | NM_004717.2 | 7 | 137182914 | 0.45 | 2.79E−06 | 0.766 |
| AGT | NM_000029.2 | 1 | 228916922 | 0.35 | 4.67E−04 | 0.709 |
| FLJ25422 | NM_145000.2 | 5 | 36337193 | 0.7 | 5.22E−04 | 0.717 |
| SEPP1 | NM_005410.2 | 5 | 42847709 | 0.1 | 2.50E−04 | 0.768 |
| SOX10 | NM_006941.3 | 22 | 36710929 | 0.7 | 1.33E−04 | 0.773 |
| MAP3K14 | NM_003954.1 | 17 | 40724512 | 0.25 | 9.75E−04 | 0.727 |
| SOX10 | NM_006941.3 | 22 | 36710385 | 0.8 | 5.45E−04 | 0.773 |
| ACOT8 | NM_183385.1 | 20 | 43920117 | 0.4 | 3.27E−04 | 0.722 |
| MGMT | NT_008818.15 | 10 | 131155565 | 0.1 | 1.10E−05 | 0.708 |
| KCNMB1 | NM_004137.2 | 5 | 169748956 | 0.15 | 2.99E−04 | 0.716 |
| CHI3L2 | NM_001025197.1 | 1 | 111571797 | 0.5 | 6.42E−04 | 0.761 |
| COG4 | NM_015386.1 | 16 | 69116377 | 0.35 | 2.10E−06 | 0.769 |
| FAM49A | NM_030797.1 | 2 | 16711469 | 0.35 | 7.29E−04 | 0.792 |
| GPR85 | NM_018970.3 | 7 | 112514937 | 0.55 | 4.99E−05 | 0.776 |
| CCND1 | NT_078088.3 | 11 | 69170515 | 0.75 | 2.84E−04 | 0.707 |
| MGC29671 | NM_182538.3 | 17 | 4283980 | 0.5 | 9.43E−04 | 0.703 |
| LGALS1 | NM_002305.2 | 22 | 36401623 | 0.15 | 5.25E−04 | 0.698 |
| SDPR | NM_004657.4 | 2 | 192420403 | 0.15 | 6.66E−04 | 0.697 |
| GPR128 | NM_032787.1 | 3 | 101811403 | 0.35 | 3.65E−04 | 0.695 |
| NET1 | NM_005863.2 | 10 | 5478485 | 0.3 | 2.40E−04 | 0.690 |
| SLC26A5 | NM_198999.1 | 7 | 102873214 | 0.25 | 9.97E−05 | 0.685 |
| RNASE3 | NM_002935.2 | 14 | 20429783 | 0.25 | 9.33E−05 | 0.680 |
| CDKN2B | NT_008413.17 | 9 | 21995995 | 0.4 | 1.11E−04 | 0.674 |
| NUP98 | NM_139131.2 | 11 | 3776115 | 0.3 | 5.90E−04 | 0.668 |
| CYP24A1 | NM_000782.3 | 20 | 52223546 | 0.3 | 9.92E−04 | 0.668 |
| ACTL6B | NM_016188.3 | 7 | 100091850 | 0.25 | 3.02E−04 | 0.664 |
| KLK10 | NT_011109.15 | 19 | 56212071 | 0.35 | 2.21E−04 | 0.657 |
| TRPV4 | NM_147204.1 | 12 | 108755302 | 0.3 | 3.39E−04 | 0.657 |
| CX36 | NM_020660.1 | 15 | 32834052 | 0.5 | 2.68E−04 | 0.654 |
| TRIM58 | NM_015431.2 | 1 | 246087435 | 0.6 | 2.09E−06 | 0.652 |
| GRIP1 | XM_934800.1 | 12 | 65359036 | 0.7 | 5.99E−05 | 0.647 |
| PHLDA2 | NM_003311.3 | 11 | 2906668 | 0.25 | 4.58E−04 | 0.647 |
| PON1 | NM_000446.3 | 7 | 94791589 | 0.45 | 6.94E−04 | 0.644 |
| SLC2A2 | NM_000340.1 | 3 | 172228723 | 0.15 | 1.70E−04 | 0.635 |
| TNF | NM_000594.2 | 6 | 31651544 | 0.5 | 7.18E−05 | 0.633 |
| FLJ23657 | NM_178497.2 | 4 | 76700323 | 0.75 | 7.36E−04 | 0.628 |
| C1orf176 | NM_022774.1 | 1 | 40747052 | 0.6 | 9.98E−04 | 0.628 |
| FLJ32447 | NM_153038.1 | 2 | 222870015 | 0.55 | 2.37E−04 | 0.628 |
| HOXA11 | NM_005523.4 | 7 | 27191747 | 0.75 | 3.27E−05 | 0.625 |
| LY6K | NM_017527.2 | 8 | 143778399 | 0.55 | 7.00E−04 | 0.624 |
| HMG20B | NM_006339.1 | 19 | 3524751 | 0.45 | 3.35E−05 | 0.621 |
| KHDRBS2 | NM_152688.1 | 6 | 63054656 | 0.1 | 3.55E−04 | 0.617 |
| WT1 | NT_009237.17 | 11 | 32407167 | 0.4 | 1.00E−03 | 0.613 |
| TFF2 | NM_005423.3 | 21 | 42644424 | 0.5 | 9.25E−04 | 0.607 |
| ZNF542 | NM_194319.1 | 19 | 61571383 | 0.15 | 5.36E−04 | 0.599 |
| ZSCAN1 | NM_182572.2 | 19 | 63236961 | 0.3 | 3.22E−04 | 0.597 |
| ZNF540 | NM_152606.2 | 19 | 42733963 | 0.55 | 1.64E−04 | 0.597 |
| HBZ | NM_005332.2 | 16 | 142482 | 0.65 | 6.96E−04 | 0.596 |
| GPR92 | NM_020400.4 | 12 | 6615553 | 0.45 | 2.44E−04 | 0.590 |
| HOXA9 | NM_152739.2 | 7 | 27171639 | 0.4 | 6.73E−04 | 0.588 |
| KCNA4 | NM_002233.2 | 11 | 29994886 | 0.4 | 8.66E−04 | 0.580 |
| RAC2 | NM_002872.3 | 22 | 35970196 | 0.2 | 2.22E−04 | 0.575 |
| CYP1B1 | NM_000104.2 | 2 | 38156723 | 0.5 | 2.65E−04 | 0.575 |
| FUT3 | NM_000149.1 | 19 | 5802504 | 0.4 | 9.63E−04 | 0.570 |
| GCET2 | NM_152785.3 | 3 | 113334846 | 0.2 | 2.03E−04 | 0.567 |
| MEGF10 | NM_032446.1 | 5 | 126654456 | 0.05 | 6.42E−04 | 0.562 |
| GRK1 | NM_002929.2 | 13 | 113369467 | 0.75 | 8.07E−04 | 0.550 |
| GPX5 | NM_001509.1 | 6 | 28601580 | 0.25 | 7.97E−04 | 0.546 |

C No. = Chromosome number.

### Example 2

### Methylation Status of the DGKI and SDPR Promoters

### Patients and Methods

### Patients and Tissue Samples.

The multi-center retrospective cohort used included patients with the following inclusion criteria: (1) Adult patients aged 18 years or more; (2) Pathological diagnosis of a glioblastoma (WHO grade IV); (3) Detailed clinical information at diagnosis and during follow-up; (4) Treatment with radiotherapy and concurrent/adjuvant temozolimide following surgery (standard “STUPP regimen”); (5) Available tumor tissue with informed consent in accordance with the French regulations and the Helsinki Declaration. DNAs were extracted with the NucleoSpin Tissue Kit (Macherey Nagel) according to the manufacturer's instructions. The quality of DNA samples was assessed by electrophoresis in a 1% agarose gel.

### Mid-Plex Custom DNA Methylation Profiling.

DNA methylation profiling was performed using the VeraCode GoldenGate Methylation technology (Illumina Inc.). A custom panel of 96 CpG sites, located within the promoter of 53 genes, has been profiled. This panel included 55 CpG sites corresponding to or located near (±50 base pairs) CpG sites previously associated with OS (see Example 1). An additional 41 CpG sites, distributed along the promoter region of CCND1, DGKI, FNDC3B, FSD1, MGMT, SOX10, TBX3, and ZNFN1A3 were added. DNA from glioblastomas and from control brains were bisulfite-modified using the EZ DNA methylation kit (Zymo Research) and hybridized according to the manufacturer's instructions. For each interrogated CpG site, the β-value is a quantitative measure of DNA methylation level. Missing values were imputed by nearest neighbor averaging (impute R package). For the 55 CpG sites quantified with both Infinium and VeraCode GoldenGate technologies, the observed correlations (n=50, r>0.9) demonstrated the consistency of the two technologies.

### MGMT Pyrosequencing.

Pyrosequencing of five CpG sites from the MGMT promoter was performed with the PyroMark Q96 CpG MGMT kit (Qiagen), according to the manufacturer's protocol. The methylation percentage of MGMT promoter is the mean value of the five CpG sites tested.

### IDH1 Mutation.

Tumor DNAs were screened for somatic mutations of IDH1 codon 132, by exon 4 PCR amplification and direct sequencing, as previously described (see Example 1).

### Statistical Analysis.

Overall survival (OS) and progression-free survival (PFS) were estimated using the Kaplan-Meier method. Comparisons between survival groups were performed using the log-rank test. For each CpG site, the β-value optimal threshold for the identification of at least 24 patients displaying hypo-methylation (β-value≦threshold) or hyper-methylation (β-value>threshold) was determined by selecting the best stratification p-value. Cox proportional hazard models gave estimates for the hazard ratios (HR). Adjusted p-values were calculated by controlling for the false discovery rate (FDR) with the Benjamini & Hochberg (BH) procedure. All tests were stratified for age, pre-operative Karnofsky Performance Status (KPS), and center. Analyses were carried out with the survival R package.

### CpG Site Classification.

CpG site classification was performed using non-negative matrix factorization (NMF) method. This classification method allows the identification of highly stable CpG sites clusters based on their methylation profiles. The optimal number of clusters (factorization rank) is based on the cophenetic correlation coefficient, the dispersion coefficient (overfitting parameter), and the rank estimation procedure applied to the randomized data (Brunet et al., Proc Natl Acad Sci U.S.A, 2004, 101: 4164-4169). This number determines the actual number of independent CpG sites optimally stratifying the cohort.

### Results

### Patients.

Over a nine years period (2001-2010), 233 patients treated in the departments of neurosurgery/neuro-oncology of Paris-Salpêtrière (n=153), Rennes (n=58) and Angers (n=22) University Hospitals fulfilled the inclusion criteria. Their demographic and clinical characteristics are summarized in Table 4.

TABLE 4
Patients demographic and clinical characteristics.

| Characteristics | All patients (n = 233) | |
|---|---|---|
| Age - yr | | |
| Median | 59 | |
| Range | 28-88 | |
| Age - no. (%) | | |
| ≦50 yr | 63 | (27) |
| >50 yr | 170 | (73) |
| Sex - no. (%) | | |
| Male | 130 | (56) |
| Female | 103 | (44) |
| Karnofsky performance status (%) | | |
| Median | 80 | |
| Range | 40-100 | |
| ND - no. | 12 | |
| Karnofsky performance status - no. (%) | | |
| ≦70 | 28 | (12) |
| >70 | 193 | (83) |
| ND - no. | 12 | (5) |
| Extent of surgery - no. (%) | | |
| Biopsy | 25 | (11) |
| Partial resection | 83 | (36) |
| Complete resection | 116 | (50) |
| ND | 9 | (4) |
| IDH1 mutational status - no. (%) | | |
| Wild-type | 216 | (93) |
| Mutated | 12 | (5) |
| ND | 5 | (2) |
| MGMT methylation status (*) - no. (%) | | |
| Unmethylated | 81 | (35) |
| Methylated | 152 | (65) |
| Overall survival - mo | | |

| | |
|---|---|
| Median | 19.1 |
| 95% CI | 17.1-22.5 |
| Progression-free survival - mo | |
| Median | 10.8 |
| 95% CI | 10-12.6 |

(*) probe MGMG_1 overlapping the sequence tested by pyrosequencing (PyroMark Q96 CpG MGMT kit). Beta-value cut-off = 5%.

### Survival Analysis According to Age, KPS, and IDH1 Status.

Univariate Cox analyses showed that age, KPS, and IDH1 mutational status of patients were significantly associated with OS. Age and IDH1 mutational status of patients were also significantly associated with PFS (Table 5). As previously reported, there was a strong association between IDH1 mutational status and MGMT methylation status (10 of the 12 IDH1 mutated tumors were MGMT methylated, chi-square test p-value <1·10⁻⁴) (Sanson et al., J Clin Oncol, 2009, 27: 4150-4154).

TABLE 5
Univariate Cox analyses restricted to the clinico-biological
factors significantly associated with OS or PFS.

| | |
|---|---|
| OS | PFS |

| Variable | HR | p-value | HR | p-value |
|---|---|---|---|---|
| Age (<50 yr vs ≧50 yr) | 0.5 | 1.2e−04 | 0.5 | 1.2e−05 |
| KPS (<70 vs ≧70) | 1.8 | 1.3e−02 | 1.3 | ns |
| IDH1 (wt vs mut) | 17.1 | 4.7e−03 | 12.2 | 4.5e−04 |
| MGMT (UM vs M.) | 2.3 | 1.5e−06 | 2.0 | 5.9e−06 |

(yr = year; HR = Hazard Ratio, wt: wild-type; mut: mutated; UM: unmethylated; M: methylated; ns: non significant)

### Survival Analysis According to CpG Sites and Clustering.

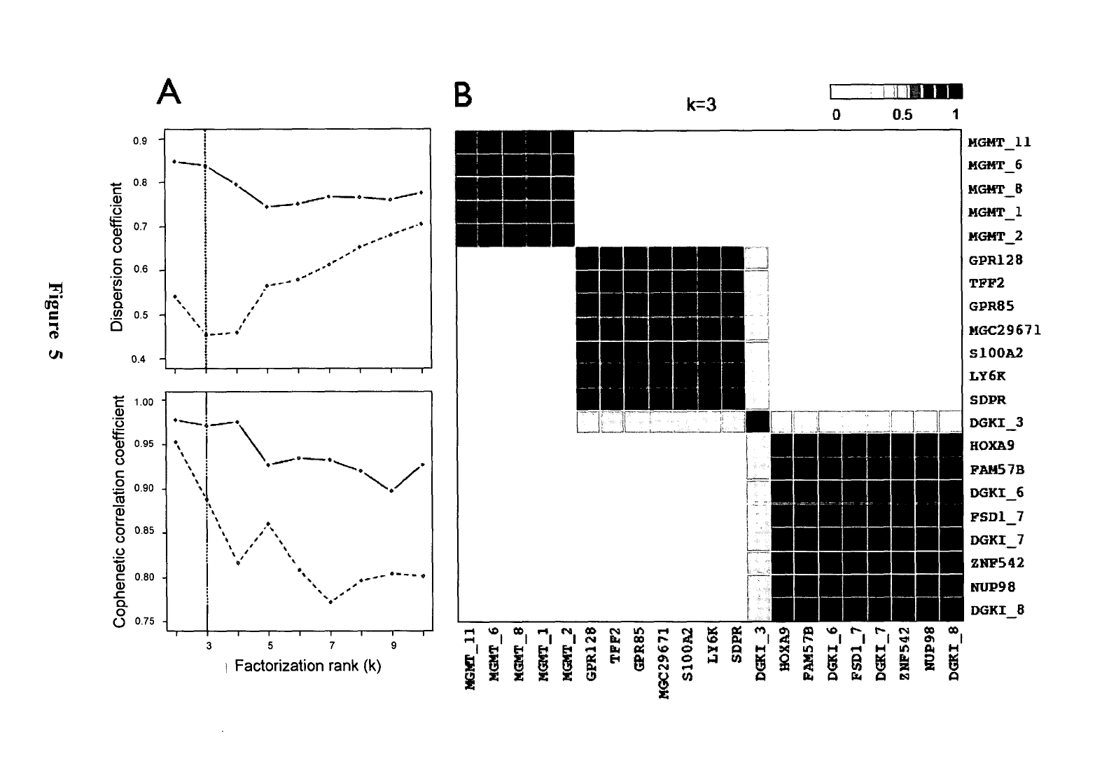

Univariate Cox analyses identified 21 CpG sites significantly associated with OS (corrected p-value ≦0.01; called “survival-associated CpG sites”). These sites had a high predictive power (absolute univariate z score greater than 3). The NMF classification clustered these 21 CpG sites into three stable classes according to their methylation profile (FIG. 5). Thus, a stratification model of three independent CpG sites was expected to capture the methylome heterogeneity of the cohort.

### Survival Analysis According to :G:T CpG Sites.

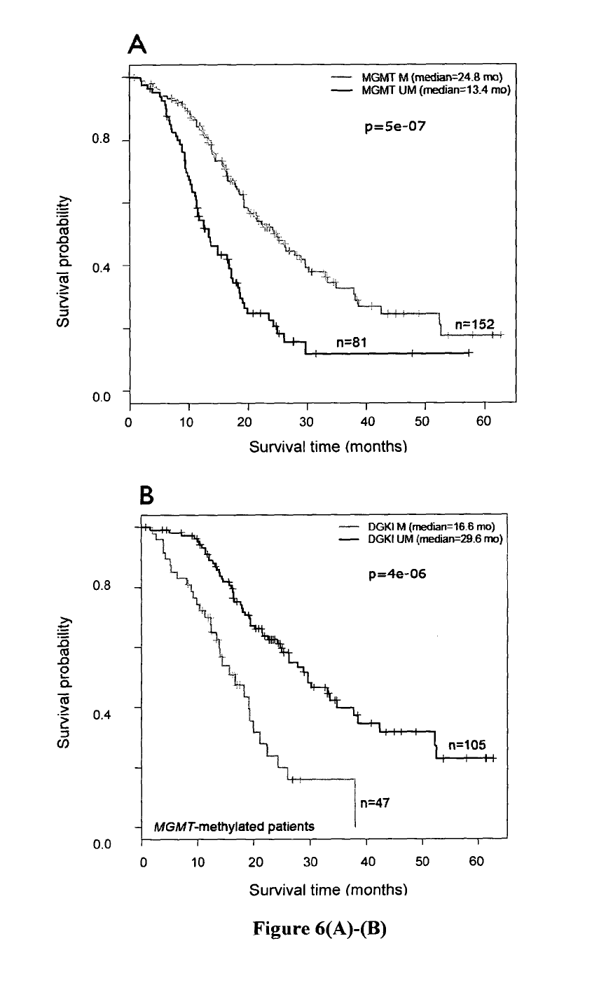

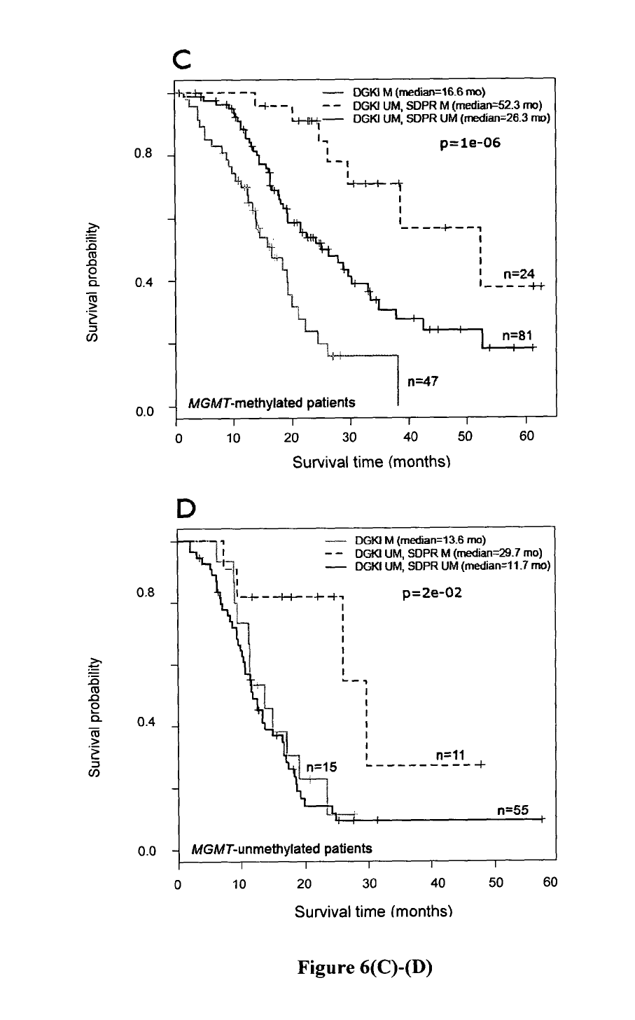

On the 12 CpG sites located within the MGMT promoter, five were survival-associated CpG sites. They all clustered in the same NMF class. Among them, the MGMT_{—}1 CpG site (FIG. 6A) was also tested by pyrosequencing, a technique previously shown to allow a good prediction of OS in glioblastoma patients receiving the standard treatment (Karayan-Tapon et al., J Neurooncol, 2010, 97: 311-322).

The methylation status (methylated/unmethylated) of this CpG site was identical for all the 50 patients studied using both techniques (data not shown). Thus, MGMT_{—}1 was used as a reference for MGMT methylation status assessment in this study. It was also considered as the first independent CpG site of the expected stratification model.

### Stratification of MGMT-Methylated Patients.

To refine the conventional MGMT stratification and identify patients who are not responding to the standard treatment, the analysis was focused on MGMT-methylated patients. Among the survival associated CpG sites, 14 CpG sites were significantly associated with OS (corrected p-value≦0.01). Among these 14 CpG sites, the multivariate Cox 2-CpG model DGKI_{—}7+SDPR was the model best associated with OS (p-value=1·10⁻⁶).

DGKI_{—}7 was the CpG site best associated with OS in univariate analysis (HR=0.3, 95% CI=0.2 to 0.5, corrected p-value=1·10⁻⁴ and z-score=−4.4). It stratified the MGMT-methylated patients into two groups with significant difference in OS (16.6 months vs. 29.6 months, p-value=4·10⁻⁶, FIG. 6B). The OS of MGMT-methylated and DGKI-methylated patients (n=47) was not significantly different from the OS of MGMT-unmethylated patients (16.6 months vs. 13.4 months, p-value=0.9). The same stratification was observed for PFS (10.2 months vs. 8.9 months, p-value=0.3).

The 2-CpG model DGKI_{—}7+SDPR stratified the MGMT-methylated patients into three groups with significant difference in OS (FIG. 6C): i) DGKI-methylated patients whose survival (16.6 months) was not affected by the SDPR status, ii) DGKI-unmethylated and SDPR-unmethylated patients with a OS of 26.3 months, and iii) a striking DGKI-unmethylated and SDPR-methylated group with a remarkably long OS (52.3 mo). The same stratification was observed for PFS (median PFS=10.2, 14.3, and 26.5 months, respectively, p-value=4·10⁻⁵).

### Stratification of MGMT-Unmethylated Patients.

The stratification of MGMT-unmethylated patients by the 2-CpG model identified two groups with significant difference in OS (FIG. 6D). DGKI-unmethylated and SDPR-methylated patients had a median OS significantly better than that of the other MGMT-unmethylated patients (29.7 months vs. 12.5 months, p-value=9·10⁻³). The same stratification was observed for PFS (20.4 months vs. 8.8 months, p-value=8·10⁻³).

### Patient Tri-Methylation Category.

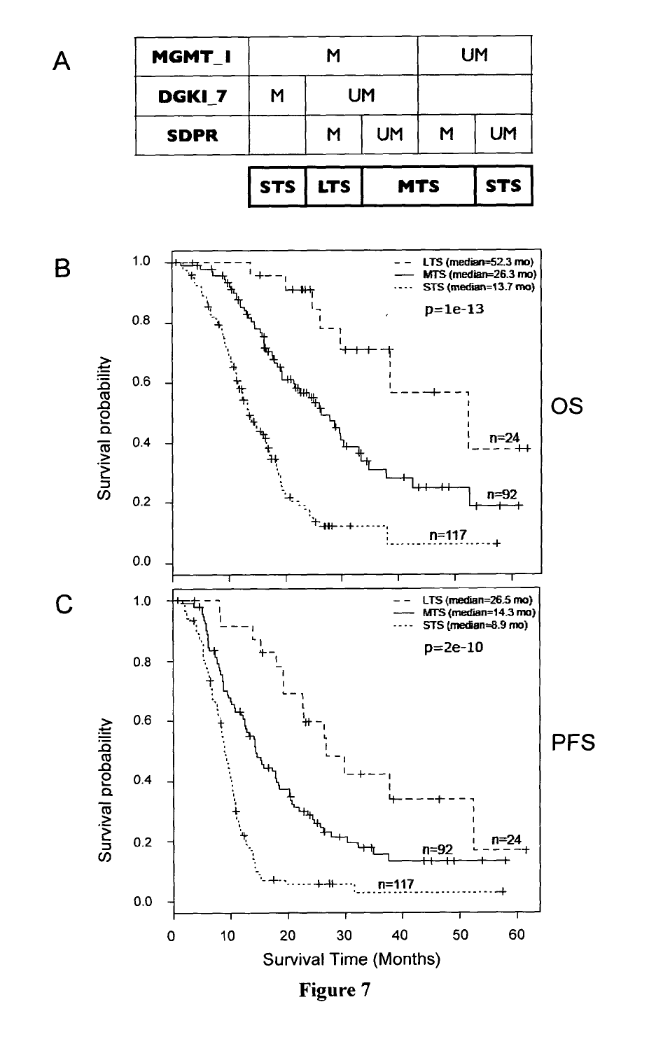

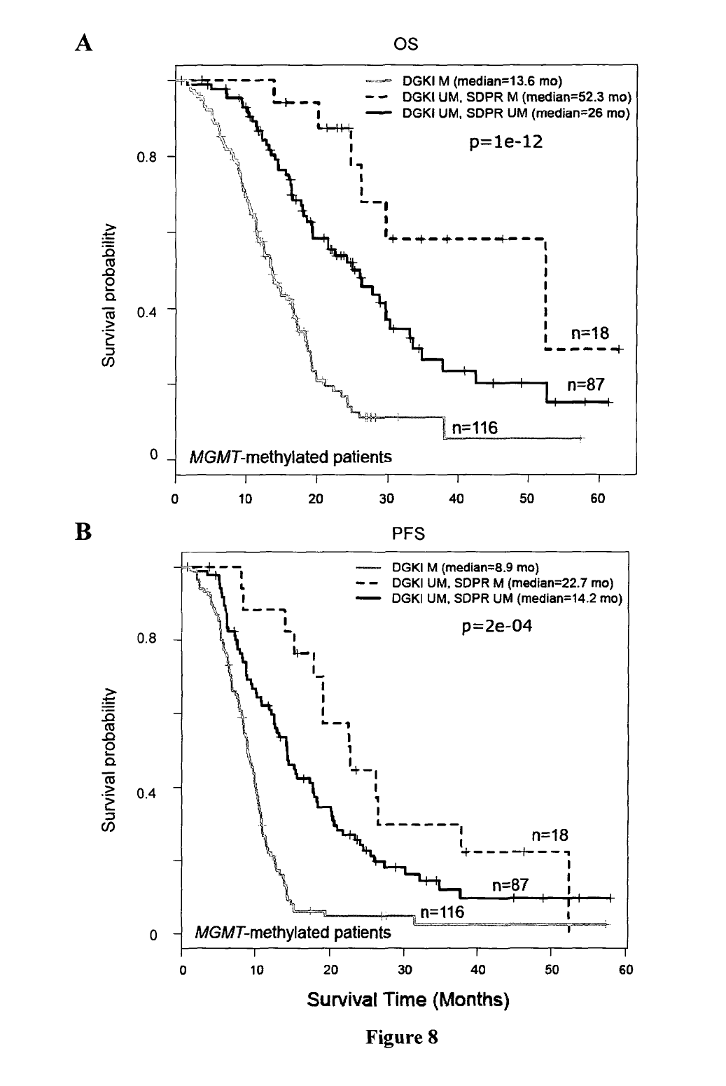

Each patient tri-methylation category was defined by the combination of its tumor methylation status (methylated/unmethylated) for MGMT, DGKI and SDPR (FIG. 7A). The tri-methylation categories were associated with significant differences in OS and named in a convenient manner: “short-term survival” or STS, “mid-term survival” or MTS, and “long-term survival” or LTS (FIGS. 7B and 7C). In a multivariate Cox model including MGMT methylation status, IDH1 mutational status, age, KPS, and center, the patient tri-methylation category was the best variable associated with OS(HR=2.6, 95% CI=1.9 to 3.7, p-value=3·10⁻⁸). Similar results were observed for PFS(HR=2.2, 95% CI=1.6 to 2.9, p-value=1·10⁻⁷).

### Discussion

In this study, quantitative DNA methylation measurements of 61 putative di-nucleotide CpG sites were performed on 233 glioblastoma patients treated with the standard Stupp protocol. In addition to the MGMT promoter methylation status, two highly robust and relevant prognostic epigenic markers for glioblastoma patient stratification were identified, namely the promoter methylation status of DGKI and SDPR. Glioblastoma patient “tri-methylation category” was defined as a combination of the promoter methylation status of MGMT, DGKI and SDPR.

In the entire cohort used, the tri-methylation category of a patient was a strong predictor of outcome and was an independent prognostic marker, having the best prognostic value in a multivariate model including known risk factors in glioblastoma (MGMT methylation status, IDH1 mutational status, age, and KPS). Consistent with previously published data (Stupp et al., Lancet Oncol, 2009, 10: 459-466), the methylation status of the MGMT promoter in the present cohort identified patients with significantly longer survival. However, in about one third of them (31%), a methylation of DGKI promoter indicated a poorer prognosis, similar to patients with an unmethylated MGMT status. The methylation status of DGKI promoter had no prognostic value for MGMT-unmethylated patients.

Conversely, a methylated status of SDPR promoter was associated with better outcome for both MGMT-methylated and -unmethylated patients. Particularly, it identified MGMT-methylated and DGKI-unmethylated patients with a very long OS, exceeding four years. These long-term survivors represented 16% of the MGMT-methylated patients. This stratification of glioblastoma patients was still valid when the cohort was restricted to the 221 patients with an IDH1 wild-type tumor (p-value=1·10⁻¹²). Although it was not specifically evaluated, it is estimated that the inclusion of the recently described “glioma-CpG island methylator phenotype (G-CIMP)” would not affect the present analysis, as this rare phenotype is very tightly associated with IDH1 mutation (Noushmehr et al., Cancer Cell, 2010, 17: 510-522).

The Non-negative Matrix Factorization classification identified three stable NMF classes of survival associated CpG sites according to their methylation profile. Each class included one of the three prognostic markers MGMT_{—}1, DGKI_{—}7, and SDPR. This suggests that these markers summarize the methylome heterogeneity of the cohort and that the methylation status of MGMT, DGKI, and SDPR optimally stratifies patients. All the survival associated CpG sites of the MGMT promoter clustered in the same NMF class. In Example 1 (see also Etcheverry et al., BMC Genomics, 2010, 11: 701), MGMT methylation and expression levels were inversely correlated (r=−0.7, p-value=5·10⁻⁷), confirming the epigenetic regulation of MGMT expression. Methylation of the MGMT gene promoter is therefore believed to regulate the expression and the effect of this DNA repair protein on the resistance to alkylating agents (Martinez et al., Neurobiol Dis, 2010, 39: 40-46). Despite their prognostic importance, the role of DGKI and SGPR as well as the functional consequences of their methylation status in glioblastoma are much less clear. Some data suggest that these genes could be involved in the oncogenesis and treatment response of glioblastoma. DGKI (diacylglycerol kinase, iota) regulates Ras signaling, an oncogenic pathway frequently altered in glioblastoma (The Cancer Genome Atlas Network: Comprehensive genomic characterization defines human glioblastoma genes and core pathways. Nature, 2008, 455: 1061-1068). SDPR (serum deprivation response) is required for stable expression of CAV1 (caveolin 1) (Hansen et al., Nat Cell Biol, 2009, 11: 807-814). Interestingly, the interaction of CAV1 and EGFR (epidermal growth factor receptor) has been characterized in glioblastoma (Abulrob et al., Oncogene, 2004, 23: 6967-6979) and was associated with poor prognosis in supratentorial ependymomas (Senetta et al., Neuro Oncol, 2011, 13: 176-183). Furthermore, treatment combining an anti-EGFR compound with temozolomide increases CAV1 expression, which in turn impairs glioblastoma cell invasiveness (Bruyere et al., Transl Oncol, 2011, 4: 92-100). However, preliminary data from the Applicants' laboratory did not show anti-correlation between methylation and expression levels for DGKI and SDPR (data not shown). An attractive hypothesis is that the methylation status of DGKI and SDPR may be the hallmark of a concerted epigenetic mechanism affecting numerous genes. Indeed, DGKI_{—}7 and SDPR belong to heterogeneous NMF classes in terms of gene composition. Further studies on this issue are clearly needed to confirm the hypothesis.

In summary, the methylation status of MGMT, DGKI and SDPR appears to encapsulate the glioblastoma methylome heterogeneity and to improve the conventional MGMT stratification of GBM patients receiving standard treatment. This stratification could be of help to refine patient recruitment and interpretation of clinical trials.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. An in vitro method for providing a prognosis for a patient diagnosed with glioblastoma, the method comprising steps of:
determining, in a biological sample obtained from the patient, the methylation status of:
the DGKI promoter and the MGMT promoter, or
the DGKI promoter, the SDPR promoter and the MGMT promoter,
to obtain a gene promoter methylation pattern for the sample, and
based on the gene promoter methylation pattern obtained, providing a prognosis for the patient, wherein the prognosis comprises the survival outcome of the patient, if the patient were to be treated according to the Stupp protocol.

2. The method according to claim 1, wherein:
the simultaneous hypermethylation of the MGMT promoter and hypermethylation of the DGKI promoter, or
the simultaneous hypomethylation of the MGMT promoter and hypomethylation of the SDPR promoter,
is indicative of a short-term survival outcome for the patient, if the patient were to be treated according to the Stupp protocol.

3. The method according to claim 1, wherein:
the simultaneous hypermethylation of the MGMT promoter, hypomethylation of the DGKI promoter and hypomethylation of the SDPR promoter, or
the simultaneous hypomethylation of the MGMT promoter and hypermethylation of the SDPR promoter,
is indicative of a medium-term survival outcome for the patient, if the patient were to be treated according to the Stupp protocol.

4. The method according to claim 1, wherein:
the simultaneous hypermethylation of the MGMT promoter, hypomethylation of the DGKI promoter and hypermethylation of the SDPR promoter
is indicative of a long-term survival outcome for the patient, if the patient were to be treated according to the Stupp protocol.

5. The method according to claim 1 further comprising a step of prescribing a treatment to the patient based on the prognosis provided.

6. The method according to claim 5, wherein prescribing a treatment to the patient comprises: prescribing a treatment according to the Stupp protocol, prescribing an alternative treatment to the Stupp protocol, or including the patient in a clinical trial for glioblastoma.

7. An in vitro method for providing a prognosis for a patient diagnosed with glioblastoma, the method comprising steps of:
determining, in a biological sample obtained from the patient, the methylation status of a promoter of at least one gene selected from the group consisting of TBX3, FSD1, FNDC3B, DGKI, AGT, FLJ25422, SEPP1, SOX10, MAP3K14, SOX10, ACOT8, KCNMB1, CHI3L2, COG4, FAM49A, GPR85, CCND1, MGC29671, LGALS1, SDPR, GPR128, NET1, SLC26A5, RNASE3, CDKN2B, NUP98, CYP24A1, ACTL6B, KLK10, TRPV4, CX36, TRIM58, GRIP1, PHLDA2, PON1, SLC2A2, TNF, FLJ23657, C1orf176, FLJ32447, HOXA11, LY6K, HMG20B, KHDRBS2, WT1, TFF2, ZNF542, ZSCAN1, ZNF540, HBZ, GPR92, HOXA9, KCNA4, RAC2, CYP1B1, FUT3, GCET2, MEGF10, GRK1, GPX5, and any combination thereof,
to obtain a gene promoter methylation pattern for the sample, and
based on the gene promoter methylation pattern obtained, providing a prognosis for the patient, wherein the prognosis comprises the response of the patient to the treatment, if the patient were to be treated according to the Stupp protocol.

8. The method according to claim 7, wherein hypomethylation of SOX10 promoter is indicative of a patent who would be responsive to the Stupp treatment, if the patient were to be treated according to the Stupp protocol.

9. The method according to claim 7, wherein the simultaneous hypermethylation of the MGMT promoter and one or more of:
hypomethylation of the FNDC3B promoter,
hypermethylation of the TBX3 promoter,
hypermethylation of the DGKI promoter, and
hypermethylaltion of the FSD1 promoter,
is indicative of a patent who would be non-responsive to the Stupp treatment, if the patient were to be treated according to the Stupp protocol.

10. The method according to claim 7 further comprising a step of prescribing a treatment to the patient based on the prognosis provided.

11. The method according to claim 10, wherein prescribing a treatment to the patient comprises: prescribing a treatment according to the Stupp protocol, prescribing an alternative treatment to the Stupp protocol, or including the patient in a clinical trial for glioblastoma.

12. The method according to claim 1, wherein the biological sample is genomic DNA extracted from a fresh tissue sample, a frozen tissue sample or a fixed, paraffin-embedded tissue sample.

13. The method according to claim 12, wherein the tissue sample is glioblastoma tissue obtained during surgery.

14. The method according to claim 12, wherein determining the methylation status is performed after sodium bisulfite conversion of the extracted genomic DNA and comprises using direct sequencing, pyrosequencing, Combined Bisulfite Restriction Analysis (COBRA), Methylation-Sensitive Single-Nucleotide Primer Extension (MS-SnuPE), Methylation-Sensitive Melting Curve Analysis or (MS-MSA), Methylation-Sensitive High-Resolution Melting (MS-HRM), MALDI-TOF mass spectrometry, HeavyMethyl, methylation specific PCR (MSP), MethylLight, Melting curve Methylation Specific PCR (McMSP), Sensitive Melting Analysis after Real-Time MSP (SMART-MSP), Methylation-Specific Fluorescent Amplicon Generation (MS-FLAG) or any combination thereof.

15. A kit for the in vitro prognosis of a glioblastoma patient treated in accordance with the Stupp protocol, the kit comprising methylation-specific PCR (MSP) primers, methylation-independent PCR (MIP) primers, or pyrosequencing primers to detect the methylation status of the DGKI promoter, the SDPR promoter and/or the MGMT promoter in genomic DNA after sodium bisulfite conversion.

16. A kit for the in vitro prognosis of a glioblastoma patient treated in accordance with the Stupp protocol, the kit comprising methylation-specific PCR (MSP) primers, methylation-independent PCR (MIP) primers, or pyrosequencing primers to detect the methylation status of at least one gene promoter in genomic DNA after sodium bisulfite conversion, wherein the gene promoter is TBX3, FSD1, FNDC3B, DGKI, AGT, FLJ25422, SEPP1, SOX10, MAP3K14, SOX10, ACOT8, KCNMB1, CHI3L2, COG4, FAM49A, GPR85, CCND1, MGC29671, LGALS1, SDPR, GPR128, NET1, SLC26A5, RNASE3, CDKN2B, NUP98, CYP24A1, ACTL6B, KLK10, TRPV4, CX36, TRIM58, GRIP1, PHLDA2, PON1, SLC2A2, TNF, FLJ23657, C1orf176, FLJ32447, HOXA11, LY6K, HMG20B, KHDRBS2, WT1, TFF2, ZNF542, ZSCAN1, ZNF540, HBZ, GPR92, HOXA9, KCNA4, RAC2, CYP1B1, FUT3, GCET2, MEGF10, GRK1, GPX5 or any combination thereof.

17. The kit according to claim 16, wherein the at least one gene promoter is the SOX10 promoter.

18. The kit according to claim 16, wherein the at least one gene promoter is the FNDC3B promoter, and/or the TBX3 promoter, and/or the DGKI promoter, and/or the FSD1 promoter.

19. The kit according to claim 16, further comprising methylation-specific PCR (MSP) primers, methylation-independent PCR (MIP) primers or pyrosequencing primers to detect the methylation status of the MGMT promoter.

20. The method according to claim 7, wherein the biological sample is genomic DNA extracted from a fresh tissue sample, a frozen tissue sample or a fixed, paraffin-embedded tissue sample.

21. The method according to claim 20, wherein the tissue sample is glioblastoma tissue obtained during surgery.

22. The method according to claim 20, wherein determining the methylation status is performed after sodium bisulfite conversion of the extracted genomic DNA and comprises using direct sequencing, pyrosequencing, Combined Bisulfite Restriction Analysis (COBRA), Methylation-Sensitive Single-Nucleotide Primer Extension (MS-SnuPE), Methylation-Sensitive Melting Curve Analysis or (MS-MSA), Methylation-Sensitive High-Resolution Melting (MS-HRM), MALDI-TOF mass spectrometry, HeavyMethyl, methylation specific PCR (MSP), MethylLight, Melting curve Methylation Specific PCR (McMSP), Sensitive Melting Analysis after Real-Time MSP (SMART-MSP), Methylation-Specific Fluorescent Amplicon Generation (MS-FLAG) or any combination thereof.
